(19) United States
(12) Patent Application Publication
(10) Pub. No.: US 2009/0246153 A1
(43) Pub. Date: Oct. 1, 2009

(54) COSMETIC COMPOSITIONS FOR THE INHIBITION OF REACTIVE OXYGEN SPECIES

(75) Inventor: Dale Kern, Hyde Park., UT (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Nu Skin International, Inc., Provo, UT (US)

(21) Appl. No.: 12/058,245

(22) Filed: Mar. 28, 2008

(51) Int. Cl. A61K 8/00; A61K 36/00; A61K 36/79; A61Q 17/04; A61Q 19/08; A61K 31/015; A61K 31/122

(52) U.S. Cl. 424/59; 424/725; 424/753; 514/763; 514/691

(57) ABSTRACT

A composition and method having anti-aging properties is provided. The composition comprises an arNOX inhibitory agent present in a natural plant extract and is useful topically as a cosmetic. The symptoms of aging including lines, wrinkles, hyperpigmentation, dehydration, loss of elasticity, angioma, dryness, itching, telangietasias, actinic purpura, seborrheic keratoses and actinic keratoses. The invention may be used multiple times a day without deleterious effects.

### FIELD OF THE INVENTION

The invention relates to extracts of natural products useful in sequestering serum aging factors that may be administered internally or topically. More particularly, the invention relates to agents and compositions thereof for use cosmetically to inhibit or ameliorate aging-related oxidation and methods for their use as skin care products.

### BACKGROUND OF THE INVENTION

The plasma membrane NADH oxidase (NOX) is a unique cell surface protein with hydroquinone (NADH) oxidase and protein disulfide-thiol interchange activities that normally responds to hormone and growth factors. NOX (or CLOX) are a family of growth related proteins that are associated with aging cells. A hormone-insensitive and drug-responsive form of the NOX designated tNOX has been described that is specific for cancer cells. For example, see U.S. Pat. No. 5,605,810, which is incorporated herein by reference in its entirety.

The aging-related isoform of NADH oxidase (arNOX) is a member of this family of proteins. The circulating form of arNOX increases markedly in human sera and in lymphocytes of individuals, especially until the age of 65. The arNOX protein is uniquely characterized by an ability to generate superoxide radicals, which may contribute significantly to aging-related changes including atherogenesis and other action-at-a-distance aging phenomena. Activity of arNOX in aging cells and in sera has been described previously. See, for example, PCT Pub. App. No. WO 00/57871, which is incorporated by reference in its entirety herein.

This model of the effects of arNOX is consistent with the Mitrochondrial Theory of Aging, which holds that during aging, increased reactive oxygen species in mitochondria cause mutations in the mitochondrial DNA and damage mitochondrial components, resulting in senescence. The mitochondrial theory of aging proposes that accumulation of spontaneous somatic mutations of mitochondrial DNA (mtDNA) leads to errors of mtDNA encoded polypeptide chains. (Manczak M et al., J Neurochem. 2005 February; 92(3):494-504). These errors, occurring in mtDNA encoded polypeptide chains, are stochastic and randomly transmitted during mitochondrial and cell division. The consequence of these alterations is defective oxidative phosphorylation. Respiratory chain defects may become associated with increased oxidative stress amplifying the original damage (Ozawa, 1995, Biochim. Biophys. Acta 1271:177-189; and Lenaz, 1998, Biochim. Biophys. Acta 1366:53-67). In this view, therefore, mutated mitochondrial DNA, despite being present only in very small quantities in the body, may be the major generator of oxidative stress.

Where accumulation of somatic mutations of mtDNA leads to defective oxidative phosphorylation a plasma membrane oxido-reductase (PMOR) system has been suggested to augment survival of mitochondrially deficient cells through regeneration of oxidized pyridine nucleotide. (de Grey, 1997, BioEssays 19:16 1-166; de Grey, 1998, Anti-Aging Med. 1:53-66; Yoneda et al, 1995, Biochem. Biophys. Res. Comm, 209:723-729; Schon et al., 1996, Cellular Aging and Cell Death, Wiley and Sons, New York, pp. 19-34; Ozawa, 1997, Physiol. Rev. 77:425-464; and Lenaz, 1998, BioFactors 8:195-204). A model to link accumulation of lesions in mtDNA to extracellular responses, such as the oxidation of lipids in low density lipoprotein (LDLs) and the attendant arterial changes, was first proposed with rhoₒ cells (Larm et al., 1994, Biol. Chem. 269:30097-30100; Lawen et al., 1994, Mol. Aspects. Med. 15:s13-s27; de Grey, 1997, BioEssays 19:161-166; and de Grey, 1998, Anti-Aging Med. 1:53-66). Similar studies have been conducted with transformed human cells in culture. (Vaillant et al., 1996, Bioenerg. Biomemb. 28:53 1-540).

Under conditions where plasma membrane oxidoreductase (PMOR) is overexpressed electrons are transferred from NADH to external acceptors by a defined electron transport chain, resulting in the generation of reactive oxygen species (ROS) at the cell surface. Such cell surface-generated ROS may then propagate an aging cascade originating in mitochondria to both adjacent cells as well as to circulating blood components such as low density lipoproteins. See PCT Pub. App. No. WO 00/57871 incorporated by reference herein in its entirety.

Consequently, there is a need to find agents that reduce the ability of arNOX to generate reactive oxygen species (ROS) for the purposes of reducing or treating the resultant physiological conditions, such as oxidation of lipids in low density lipoprotein (LDLs) and attendant arterial changes. The arNOX activity of aging cells has been shown to be inhibited by naturally occurring agents such as, co-enzyme Q (ubiquinone). See PCT Pub. App. Nos. WO 00/57871, WO 01/72318, and WO 01/72319, the disclosures of which are incorporated herein by reference in their entirety. However, the use of co-enzyme Q is not completely satisfactory for several reasons: it is costly, it oxidizes easily losing its efficacy, and preparations containing coenzyme Q must be specially packaged to prevent loss of function. Thus, while some agents and methods currently exist, which may inhibit arNOX activity, challenges still exist. Accordingly, it would be an improvement in the art to augment or even replace previously disclosed agents and techniques with the agents and techniques that inhibit arNOX but that are also non-toxic and naturally occurring.

The skin in particular is vulnerable to damage by reactive oxygen species. The skin is composed of two major layers. The stratum corneum, or epidermis, is the top layer and forms a protective covering for the skin and controls the flow of water and substances in and out of the skin. The dermis is the lower level of the skin and provides the strength, elasticity and the thickness to the skin. The main cell type of the dermis is fibroblasts, which are responsible for synthesis and secretion of all the dermal matrix components such as collagen, elastin and glycosaminoglycans. Collagen provides the strength, elastin the elasticity, and glycosamino-glycans the moistness and plumpness of the skin.

In addition to being damaged by reactive oxygen species the skin is subject to various damaging stressors. The skin may be damaged or abused by many factors in the environment. Some are naturally occurring such as UV radiation from the sun, wind and even mechanical insults such as cuts, scrapes and the like. Other, man-made insults also occur daily. These include the use of soaps, emulsifier-based cosmetics, hot water, organic solvents, air conditioning and central heating. Further, other insults to the skin may result from or be part of dermatological disorders or the normal aging process (chronoaging), which may be accelerated by exposure of skin various external stressors (e.g. photoaging).

Everyone's skin ages with time. In modern society, however, people live longer and the normal effects of aging have an opportunity to accumulate. Such effects may be purely cosmetic, such as the increase in wrinkles or “age spots” or they may have an impact on health such as the incidence of skin cancer due to exposure to UV light. As people age the skin becomes thinner, the connective tissue of the skin, collagen and elastin changes causing the skin to loose firmness and become dry. Also, the sweat and oil glands of the skin become less active thereby causing the skin to lose moisture and dry out. Further, blood vessels in the skin become more fragile so that they rupture and leak into the skin.

Symptoms of aging skin include dryness, itchiness, thinning or thickening of the skin, wrinkles and fine lines, areas of hyperpigmentation commonly referred to as liver spots and areas underneath the skin where blood vessels have ruptured (telangietasias).

“Anti-aging” cosmetic and medical products, treat or delay the visible signs of actual aging and weathered skin such as wrinkles, lines, sagging, hyperpigmentation and age spots are desirable. However, most cosmetic or medicinal products do not address causes of such symptoms e.g., the production and build up of arNOX related radicals derived from ROS. Accordingly, there is a demand for effective natural skin treatments and preventative compositions and methods for using the same.

### SUMMARY OF THE INVENTION

The present invention is directed to naturally occurring agents which may be administered either internally or topically which specifically inhibit arNOX and ameliorate some of its aging related effects. Such agents can take the form of isolated agents or plant extracts. Further, while arNOX inhibitory agents can be used alone, they may also be used as compositions comprising multiple arNOX inhibitory agents and/or formulations including compounds having other beneficial effects on the body. In particular, the inventors have found that by adding arNOX inhibitors to cosmetics, the inhibitors can have beneficial effects that augment the normal skin care regimen.

Therefore, in one exemplary embodiment, the invention comprises a composition useful for ameliorating the effects of aging comprising an effective amount of at least one arNOX inhibitory agent. In this exemplary embodiment, the arNOX inhibitory agent is effective in decreasing the effects of aging. In some versions, the invention further includes a cosmetically or pharmaceutically acceptable carrier. In various exemplary embodiments according to the invention, the arNOX inhibitory agent is present in a plant extract. In some embodiments, the arNOX inhibitory agent is purified from a plant extract. In various exemplary embodiments, the plant is selected from broccoli, shiitake, coleus, rosemary, lotus, artichoke, sea rose, tangerine, Oenothera biennis, astaxanthin, red orange, Schisandra chinensis, Lonicera, Fagopyrum, carrot, Narcissus tazetta or olive.

It should be appreciated that the invention can be administered in any suitable way. For example, in various exemplary embodiments, the invention can be administered topically, orally, parenterally, transdermally or rectally. In these and other exemplary embodiments, effects of aging include lines, wrinkles, hyperpigmentation, dehydration, loss of elasticity, angioma, dryness, itching, telangietasias, actinic purpura, seborrheic keratoses and actinic keratoses.

In yet another exemplary embodiment, the invention comprises a cosmetic composition for ameliorating the effects of aging comprising a cosmetically effective amount of at least one arNOX inhibitory agent wherein the arNOX inhibitory agent is effective in decreasing the effects of aging upon the skin. In one version of this exemplary embodiment, the invention includes a cosmetically acceptable carrier. In this embodiment, the carrier may include powders, emollients, lotions and liquids. In some exemplary embodiments, the arNOX inhibitory agent is derived from a plant. In particular exemplary embodiments, the plant is selected from broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta or olive.

It should be appreciated that the cosmetic composition according to this exemplary embodiment can be administered in any effective manner. For example, in some exemplary embodiments, the cosmetic composition according to the invention is applied topically, orally, parenterally, transdermally or rectally. In some exemplary embodiments, the composition is formulated as a cream, a milk, a lotion, a gel, a suspension of lipid or polymeric microspheres or nanospheres or vesicles, a soap or a shampoo.

In still other exemplary embodiments, the invention includes a cosmetic method for ameliorating the effects of aging comprising applying to the skin a cosmetic composition comprising an effective amount of an arNOX inhibitor, wherein at least one arNOX mediated effect of aging is inhibited. In some exemplary embodiments according to the invention, the arNOX inhibitor is a plant extract. In other exemplary embodiments, the arNOX inhibitor is purified from a plant extract. In various exemplary embodiments according to the invention the arNOX inhibitory agent is present in a concentration of between about 5 μg/ml to about 500 μg/ml. In other exemplary embodiments, the inhibitory agent is present in a concentration of between about 15-100 μg/ml. In some exemplary embodiments, the cosmetic composition according to the invention is applied topically, orally, parenterally, transdermally or rectally or in any other effective manner. In some exemplary embodiments, the composition is formulated as a cream, a milk, a lotion, a gel, a suspension of lipid or polymeric microspheres or nanospheres or vesicles, a soap or a shampoo.

In still other exemplary embodiments, the invention comprises a kit. In this embodiment, the kit may include a volume of an arNOX inhibitory agent and instruction for use. In various exemplary embodiments, the kit may further include a cosmetic preparation so that the arNOX inhibitory agent can be added to the cosmetic preparation prior to use.

It should be appreciated that while in some exemplary embodiments of the invention, only one arNOX inhibitory agent is used, in other exemplary embodiments more than one extract or arNOX inhibitory agent are used together. Further, it should be appreciated that in various exemplary embodiments of the invention, the one or various arNOX inhibitory agents may be applied or administered in various ways. Such as, for example, topical administration, formulated, for example as a cream, a milk, a lotion, a gel, a suspension of lipid or polymeric microspheres or nanospheres or vesicles, a soap, a shampoo or a sunscreen and in the form of a tea or capsule or any other effective manner.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be apparent from the description, as set forth hereinafter.

### DETAILED DESCRIPTION OF THE EXEMPLARY EMBODIMENTS

The invention relates to agents for sequestering serum aging factors, and methods for using the same. More particularly, the invention relates to agents and methods for using such factors, to prevent or treat disorders and complications of disorders resulting from cell damage caused by an aging-related isoform of NADH oxidase (arNOX). In one exemplary embodiment the agents of the invention comprise at least one naturally occurring arNOX inhibitor. One embodiment of the invention comprises agents that bind arNOX and inhibit the ability of arNOX to generate reactive oxygen species as well as methods of using such agents to inhibit the ability of arNOX to generate reactive oxygen species.

The invention provides pharmaceutical and/or cosmetic compositions, methods of use, and kits comprising inhibitory agents for the treatment or amelioration of disorders or effects resulting from oxidative changes in cells that result in aging by targeting an aging-related isoform of NADH oxidase (arNOX), shed into the sera by aging cells. The compositions may contain inhibitory agents extracted from plants. For example the compositions of the invention may comprise at least one broccoli product, whether alone or with other inhibition agents and inhibit the activity of an aging-related isoform of NADH oxidase shed into the sera by aging cells, wherein the other inhibitory agent may comprise extracts, for example, of shitake, lotus, artichoke, astaxanthin and the like. Of course it should be understood that such active agents or extracts can be used in combination with other arNOX inhibitors, such as, ubiquinones, extracts of Schisandra chinensis, or Lonicera japonica, or extracts of Fagopyrum cymosum, Narcissus tazetta and the like or in combination with lotions, emollients and preservatives as necessary.

As used herein the term “cosmetic” refers to a substances intended to be applied to the body for cleansing, beautifying, promoting attractiveness, or altering the appearance. As used herein the term “extract” refers to a solution obtained by steeping or soaking a substance in a solvent and removing the active ingredient. The solvent can be any suitable solvent including but not limited to alcohol, water or the like. In some instances the extract is concentrated or the solvent can be evaporated and the active ingredient resuspended or solubilized in a different solvent.

As used herein, the term “disorder” refers to any condition of a living animal or plant body or of one of its parts that impairs normal functioning comprising any ailment, disease, illness, clinical condition, pathological condition, weakened condition, unsound condition, and any abnormal or undesirable physical condition.

As used herein, the term “reactive oxygen species” refers to oxygen derivatives from oxygen metabolism or the transfer of free electrons, resulting in the formation of free radicals (e.g., superoxides or hydroxyl radicals).

As used herein, the term “antioxidant” refers to compounds that neutralize the activity of reactive oxygen species or inhibit the cellular damage done by said reactive species.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment corresponding to the formulations described in this application, which exhibit antioxidant activity, and is relatively non-toxic to the subject.

The term “therapeutic agent” refers to any molecule, compound, or treatment, preferably an antioxidant, which assists in the prevention or treatment of the disorders, or complications of disorders caused by reactive oxygen species.

The term “agent that sequesters arNOX” refers to any molecule, compound, or treatment that interacts with arNOX, thus decreasing the reaction of arNOX with other substrates and inhibits the ability of arNOX to generate reactive oxygen species.

The antioxidants, cellular components, and target proteins defined herein are abbreviated as follows:

| | |
|---|---|
| mitochondrial DNA | mtDNA |
| nicotinamide adenine dinucleotide | NADH |
| cell surface hydroquinone (NADH) oxidase with | NOX |
| protein disulfide-thiol isomerase activity | |
| NOX specific to non-cancer cells | cNOX |
| NOX specific to aged cells | arNOX |
| NOX specific to cancer cells | tNOX |
| low density lipoprotein | LDL |
| plasma membrane oxido-reductase chain | PMOR |
| ubiquinone or coenzyme Q | CoQ |
| coenzyme Q₁₀ | CoQ₁₀ |
| reactive oxygen species | ROS |

The following disclosure of the present invention is grouped into subheadings. The utilization of the subheadings is for convenience of the reader only and is not to be construed as limiting in any sense.

### THE INVENTION

The present invention is directed to naturally occurring agents which may be administered either internally or topically which specifically inhibit arNOX and ameliorate some of its aging related effects. Such agents can take the form of isolated agents or plant extracts. Further, while arNOX inhibitory agents can be used alone, they may also be used as compositions comprising multiple arNOX inhibitory agents and/or formulations including compounds having other beneficial effects on the body. In particular, the inventor has found that by adding arNOX inhibitors to cosmetics, the inhibitors can have beneficial effects that augment the normal skin care regimen.

Therefore, in one exemplary embodiment, the invention comprises a composition useful for ameliorating the effects of aging comprising an effective amount of at least one arNOX inhibitory agent. In this exemplary embodiment, the arNOX inhibitory agent is effective in decreasing the effects of aging. In some version, the invention further includes a cosmetically or pharmaceutically acceptable carrier. In various exemplary embodiments according to the invention, the arNOX inhibitory agent is present in a plant extract. In some embodiments, the arNOX inhibitory agent is purified from a plant extract. In various exemplary embodiments, the plant is selected from broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta or olive.

It should be appreciated that the invention can be administered in any suitable way. For example, in various exemplary embodiments, the invention can be administered topically, orally, parenterally, transdermally, rectally or any other effective method. In these and other exemplary embodiments, effects of aging include lines, wrinkles, hyperpigmentation, loss of hydration, loss of elasticity, decrease in collagen, angioma, dryness, itching, telangietasias, actinic purpura, seborrheic keratoses and actinic keratoses.

In yet another exemplary embodiment, the invention comprises a cosmetic composition for ameliorating the effects of aging comprising a cosmetically effective amount of at least one arNOX inhibitory agent wherein the arNOX inhibitory agent is effective in decreasing the effects of aging upon the skin. In one version of this exemplary embodiment, the invention includes a cosmetically acceptable carrier. In this embodiment, the carrier may include powders, emollients, lotions, creams, liquids and the like. In some exemplary embodiments, the arNOX inhibitory agent is derived from a plant. In particular exemplary embodiments, the plant is selected from broccoli, shitake, coleus, rosemary, lotus, artichoke, sea rose, tangerine, Oenothera biennis, astaxanthin, red orange, Schisandra chinensis, Lonicera, Fagopyrum, carrot, Narcissus tazetta or olive.

It should be appreciated that the cosmetic composition according to this exemplary embodiment can be administered in any exemplary manner. For example, in some exemplary embodiments, the cosmetic composition according to the invention is applied topically, orally, parenterally, transdermally or rectally. In some exemplary embodiments, the composition is formulated as a cream, a milk, a lotion, a gel, a suspension of lipid or polymeric microspheres or nanospheres or vesicles, a soap or a shampoo.

In still other exemplary embodiments, the invention includes a cosmetic method for ameliorating the effects of aging comprising applying to the skin a cosmetic composition comprising an effective amount of an arNOX inhibitor, wherein at least one arNOX mediated effect of aging is inhibited. In some exemplary embodiments according to the invention, the arNOX inhibitor is a plant extract. In other exemplary embodiments, the arNOX inhibitor is purified from a plant extract. In various exemplary embodiments according to the invention, the arNOX inhibitory agent is present in a concentration of between about 5 μg/ml to about 500 μg/ml. In various exemplary embodiments, the concentration of the active agent is present in a concentration of between about 15 to 100 μg/ml. In some exemplary embodiments, the cosmetic composition according to the invention is applied topically, orally, parenterally, transdermally, rectally or by any other effect method. In some exemplary embodiments, the composition is formulated as a cream, a milk, a lotion, a gel, a suspension of lipid or polymeric microspheres or nanospheres or vesicles, a soap or a shampoo.

In still other exemplary embodiments, the invention comprises a kit. In this embodiment, the kit may include a volume of an arNOX inhibitory agent and instruction for use. In various exemplary embodiments, the kit may further include a cosmetic preparation such that the arNOX inhibitory agent can be added to the cosmetic preparation prior to use.

It should be appreciated that while in some exemplary embodiments of the invention, only one arNOX inhibitory agent is used, in other exemplary embodiments more than one extract or arNOX inhibitory agent are used together. Further, it should be appreciated that in various exemplary embodiments of the invention, the one or various arNOX inhibitory agents may be applied or administered in various ways. Such as, for example, topical administration in any effective manner, such as, for example, a cream, a milk, a lotion, a gel, a suspension of lipid or polymeric microspheres or nanospheres or vesicles, a soap, a shampoo or a sunscreen and in the form of a tea or capsule or any other effective manner.

Plasma Membrane Hydroquinone (NADH) Oxidase (NOX)

The plasma membrane NADH oxidase (NOX) is a unique cell surface protein with hydroquinone (NADH) oxidase and protein disulfide-thiol interchange activities that normally responds to hormone- and growth factors. A hormone insensitive and drug-responsive form of the activity designated tNOX also has been described, which is specific for cancer cells. Evidence exists that NOX proteins, under certain conditions, are capable of the production of ROS. For example, ultraviolet light as a source of oxidative stress in cultured cells is used to initiate superoxide generation (Morré et al., 1999, Biofactors 9:179-187) (See U.S. Pat. No. 5,605,810, which is incorporated herein by reference in its entirety).

Isolation and Characterization of arNOX

The invention encompasses research related to arNOX, an aging-related isoform of the cell surface NADH oxidase, which is capable of oxidizing reduced quinones. The NOX protein is anchored in the outer leaflet of the plasma membrane (Morré, 1995, Biochem. Biophys. Acta. 1240:201-208; and DeHahn et al., 1997, Biochem. Biophys. Acta. 1328:99-108). NOX activity was shown to be shed in soluble form from the cell surface (Morré et al., 1996, Biochim. Biophys. Acta 1280:197-206). The presence of the shed form in the circulation provides an opportunity to use patient sera as a source of the NOX protein for isolation and characterization studies. A serum form of the CNOX activity specific to sera from elderly subjects (arNOX) has been identified. (PCT Pub. App. No. WO 00/57871).

The invention is based on the identification of arNOX, which is a constitutive cell surface NADH oxidase protein (cNOX) capable of oxidizing reduced quinones. The NOX proteins have been postulated to link the accumulation of lesions in mitochondrial DNA to cell surface accumulations of reactive oxygen species as one consequence of its role as a terminal oxidase in a plasma membrane electron transport chain (Morré, D. M. et al., 2000, J. Expl Biol 203:1513-1521). Cells with functionally deficient mitochondria become characterized by an anaerobic metabolism. NADH accumulated from the glycolytic production of ATP and an elevated plasma membrane electron transport activity become necessary to maintain the NAD+/NADH homeostasis essential for survival. Previous findings demonstrate that the hyperactivity of the plasma membrane electron transport system results in an NADH oxidase activity capable of cell surface generation of reactive oxygen species (Morré, D. J. et al., 1999 BioFactors 9:179-187). This would serve to propagate the aging cascade both to adjacent cells and to oxidize circulating lipoproteins.

Generally, the characteristics of aged cells includes those that express and/or shed arNOX, and include, but are not limited to, those exhibiting one or more of the following characteristics: an age-related PMOR system, the ability to generate reactive oxygen species, and have functionally defective mitochondria. One embodiment of the invention is the utilization of agents to reduce the negative effects of aging cells.

Methods of Detecting arNOX:

The invention encompasses methods for detecting cell-membrane associated arNOX and soluble arNOX in sera. See, e.g., PCT Pub. App. No. WO 00/57871, which is incorporated herein by reference in its entirety. The invention further contemplates using arNOX as a diagnostic tool when oxidative damage to cells and/or tissue is suspected. As such, arNOX in tissue, cells, or circulation may be detected. Embodiments include: detection by employing antibodies specific to arNOX, which may be conjugated to a wide variety of labels, wherein the label provides a detectable signal. For example radioisotopes, enzymes, fluorescence and the like may be utilized as labels. Examples of detection techniques comprise: detection based upon assays that recognize that sera with arNOX exhibits a higher rate of cytochrome c reduction than sera without arNOX; an assay which measures the disappearance of the ascorbate radical spectrophotometrically by measuring the absorbance at about 265 nm since arNOX reduces an electron acceptor, e.g., ascorbate radical; by measuring the reduction of NADH by arNOX using methods known in the art; assays based on the unique oscillation property of arNOX; arNOX may be detected by resistance to retinoic acid, since NOX from healthy cells is inhibited by retinoic acid and arNOX is not inhibited by retinoic acid; a method using arNOX to identify cells where mitochondrial functions are depressed and consequently, PMOR is overexpressed; and cells may be identified in the presence of overexpressed arNOX (Morré, 1998, Plasma Membrane Redox Systems and their Role in Biological Stress and Disease 121-156; Morre et al., 1999, Mol. Cell. Biochem. 200:7-13, wherein each of the referenced documents is incorporated by reference in its entirety).

Methods of Identifying Agents that Interact with arNOX:

The present invention has utilized in vitro and in vivo methods for screening for agents which target arNOX. Within the broad category of in vitro selection methods, several types of methods are likely to be particularly convenient and/or useful for screening test agents comprising: methods which measure a binding interaction between two or more components; and methods which measure the activity of an enzyme which is one of the interacting components, i.e., arNOX. See, for example, the description in Pub. App. No. WO 00/57871, the disclosure of which is incorporated herein by reference.

Binding interactions between two or more components can be measured in a variety of ways known in the art. One approach is to label one of the components with an easily detectable label, place it together with the other component(s) in conditions under which they would normally interact (e.g., ubiquinone), perform a separation step which separates bound labeled component from unbound labeled component, and then measure the amount of bound component. The test agent may be labeled with a various detectable markers, and the separation step in this type of approach can be accomplished in various ways. See, for example, Pub. App. No. WO 00/57871.

The symptoms of aging skin include dryness, itchiness, thinning or thickening of the skin, wrinkles and fine lines, areas of hyperpigmentation (called age or liver spots), and a mottled appearance. Aging skin has been shown to have a decrease in collagen and a concomitant decrease in elasticity. In addition, aging skin has increased amounts of cleaved collagen and cross-linked proteins. Superoxide radicals have been indicated in these processes. The skin may take more time to heal when injured. Blood vessels are easier to see through the thinning skin, also because they become dilated with age. These blood vessels may be visible as red dome-like formations on the skin (cherry angiomas), or as broken capillaries on the face (telangietasias). Many people develop senile or actinic purpura, which are purplish spots or patches on the skin created by small hemorrhages in the skin. Older skin has less protection against sun damage because protective cells called melanocytes decrease with age. Aging skin is also more likely to develop a variety of benign and pre-cancerous growths, such as seborrheic and actinic keratoses. Seborrheic keratoses often have a rough, brown appearance, and look like a wart. They are benign. Actinic keratoses are small, scaly growths on areas of the skin that have received sun exposure. They are an early sign of skin cancer.

The invention encompasses the use of topical administration of natural plant extracts, alone or in the form of a cream emollient, lotion or the like, to maintain skin vitality. A preferred embodiment of the invention comprises the topical administration of a cream, lotion, emollient or the like, which comprises an arNOX inhibiting extract, to the skin of patients to maintain and improve skin vitality.

Treatment of Skin

The present invention provides compositions comprising active agent(s), which prevent and/or ameliorate skin damage and associated conditions, particularly those resulting from aging and associated with arNOX. Further, the invention encompasses methods for utilizing said compositions. The stratum corneum is the layer of the skin that forms the top surface layer and serves to protect the skin while controlling moisture and the flow of substances in and out of the skin. As this barrier function is broken down, the skin suffers damaging effects, thus further contributing to premature aging. These damaging effects causing premature aging of the skin are a concern for many individuals wishing to maintain healthy, youthful looking and feeling skin. Reactive oxygen species participate in a number of destructive reactions potentially lethal to cells. Reactive oxygen species are responsible in part for deleterious cellular interactions including impairing fibroblast cells ability to produce healthy collagen and elastin. Furthermore, the skin is subject to deterioration through dermatological disorders, environmental abuse (wind, air conditioning, central heating) or through the normal aging process (chronoaging), which may be accelerated by exposure of skin to sun (photoaging).

A preferred embodiment of the invention provides naturally occurring active agents from plants for the treatment of skin. The active agents prevent and/or ameliorate skin damage and associated conditions. In one embodiment of the invention the processed plant products sequester arNOX activity. In another embodiment of the invention, the processed plant products inhibit reactive oxygen species. In another embodiment agents and methods of the invention prevent and/or improve the health of the skin. For example, the agents may improve skin tone, e.g., tautness of skin, color and appearance of pores, elasticity, hydration and/or help diminish the appearance of fine lines and visible signs of aging. In another exemplary embodiment of the invention, the agents positively affect the body's natural production of collagen and elastin. In another embodiment, the agents of the invention minimize the effects of environmental agitators such as pollution, sun, free radicals and stress.

One embodiment of the invention provides compositions, and methods for using the same, for preventing and/or ameliorating dermatological disorders and the effects thereof.

One embodiment of the invention provides composition for preventing and reducing the effects of the production of reactive oxygen species and methods for using the same. For example, the invention encompasses the use of active agents derived from plants to at least partially sequester or inhibit arNOX activity. Further, the invention contemplates the use of other synthetic and natural compounds to sequester arNOX activity.

The present invention discloses compositions, which treat the skin and delay the visible signs of actual aging and weathered skin such as wrinkles, lines, sagging, hyperpigmentation and age spots. The present invention also decreases the appearance and condition of sensitive, dry and/or flaky skin, serves to soothe red, and/or irritated skin, and treats spots, pimples, blemishes, and other skin irregularities.

The present invention advances prior art compositions by providing compositions and methods for using the same not previously disclosed. The invention provides pharmaceutical or cosmetic compositions, methods of use, and pharmaceutical or cosmetic kits for the treatment of disorders resulting from oxidative changes in cells that result in aging by targeting an aging-related isoform of NADH oxidase (arNOX), shed into the sera by aging cells. The compositions may contain agents extracted from plants. For example, the compositions of the invention may comprise at least one extract shown to inhibit arNOX activity, whether alone or with other inhibition agents and, at least partially, inhibit or block the activity of an aging-related isoform of NADH oxidase shed into the sera by aging cells. The composition may comprise ubiquinones, natural extracts or agents derived therefrom known to comprise active agents useful in inhibiting arNOX, together with other compounds known in the art to make creams, lotions, emollients and the like. Such other compounds may comprise gums, fillers, preservatives and the like.

In one embodiment a portion of, or all of these ingredients may be combined with other ingredients commonly found in anti-aging and repair serum formulations. Vehicles, other than, or in addition to water can include liquid or solid emollients, solvents, humectants, thickeners and powders. The vehicle may be from 0.1% to 99.9%, preferably from 25% to 80% by weight of the composition, and can, in the absence of other cosmetic adjuncts, form the balance of the composition. In one embodiment, the vehicle is at least 80% water, by weight of the vehicle. In another embodiment water comprises at between about 50% to 85% of the composition by weight. In yet another embodiment, water is present between about 0.1% to 55%, by weight of the composition. In other embodiments other vehicles are used in the above recited concentrations.

An oil or oily material may be present, together with an emulsifier to provide either a water-in-oil emulsion or an oil-in-water emulsion, depending largely on the average hydrophilic-lipophilic balance (HLB) of the emulsifier employed.

The inventive compositions may also include sunscreens. Sunscreens include those materials commonly employed to block ultraviolet light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the emulsions can vary depending upon the degree of protection desired from the sun's UV radiation.

Emollients may further be incorporated into cosmetic compositions of the present invention. Levels of such emollients may range from 0.5% to 50%, preferably between 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols, which may serve as emollients are linear and branched chain alkyl polyhydroxyl compounds. For example, propylene glycol, sorbitol and glycerin are preferred. Also useful may be polymeric polyols such as poly-propylene glycol and polyethylene glycol. Butylene and propylene glycol are also especially preferred as penetration enhancers.

Exemplary hydrocarbons which may serve as emollients are those having hydrocarbon chains anywhere from 12 to 30 carton atoms. Specific examples include mineral oil, petroleum jelly, squalene and isoparaffins.

Other embodiments of the compositions of the present invention comprise thickeners. A thickener will usually be present in amounts anywhere from 0.1 to 20% by weight, preferably from about 0.5% to 10% by weight of the composition. Exemplary thickeners are cross-linked polyacrylate materials available under the trademark CARBOPOL® from the B.F. Goodrich Co. Gums may be employed such as xanthan, carrageenan, gelatin, karaya, pectin and locust beans gum. Under certain circumstances the thickening function may be accomplished by a material also serving as a silicone or emollient. For instance; silicone gums in excess of 10 centistokes and esters such as glycerol stearate have dual functionality.

Powders may be incorporated into the cosmetic composition of the invention.

These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range anywhere from 0.001% up to 20% by weight of the composition.

The composition of the invention may be used for topical application to human skin, as an agent for conditioning, moisturizing and smoothing the skin, increasing the flexibility and elasticity and preventing or reducing the appearance of wrinkled, lined or aged skin. Formulations of the present invention offer a response to the loss of skin tone and promotes benefits to effectively boost hydration and firmness of the surface layer of the skin, all while working to repair the underlying layers of the skin with antioxidants and other beneficial ingredients to help diminish the appearance of fine lines and wrinkles and to restore visible tone and elasticity. In some exemplary embodiments such anti-oxidants are specifically directed to inhibit arNOX.

In one embodiment a small quantity of the composition comprised of from about 1 to 1000 ml of active agent, is applied to the skin. In a preferred embodiment, a quantity of composition comprising from about 1 to 100 ml of active agent is applied to the skin. This process may be repeated several times daily for any period of time. Preferably, the composition is applied to the skin once in the morning and once in the evening.

The topical skin care composition of the invention can be formulated as a lotion, a cream, a gel or the like. The composition can be packaged in a suitable container to suit its viscosity and intended use by the consumer. For example, a lotion or a cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

The following examples are offered by way of illustration and not by way of limitation.

### EXAMPLES

### Example 1

### Characterization of arNOX

Superoxide Production By Buffy Coats: Buffy coats, a mixture of lymphocytes and platelets. Such buffy coats are commercially available from, for example Rockland ImmunoChemicals (Gilbertsville, Pa.). The blood samples were maintained at 4° C. prior to collection and assay. Ca. 10⁷ cells were added to each assay. Cell numbers were determined using a hemocytometer.

Reduction of ferric cytochrome c by superoxide was employed as a standard measure of superoxide formation (Mayo, L. A. and Cumutte, J. (1990) Meth. Enzyme. 186, 567-575. 7. Butler, J, Koppenol, W. H. and Margollash, E. (1982) J. Biol. Chem. 257, 10747). This is a widely accepted method when coupled to superoxide dismutase inhibition for the measurement of superoxide generation. The assay consists of 150 μl serum or 40 μl buffy coats in PBSG buffer (8.06 g NaCl, 0.2 g KCl, 0.18 g Na₂HPO₄, 0.26 g KH₂PO₄, 0.13 g CaCl₂, 0.1 g MgCl₂ 1.35 g glucose dissolved in 1000 ml deionized water, adjusted to pH 7.4, filtered and stored at 4° C.) Rates were determined using an SLM Aminco DW-2000 spectrophotometer (Milton Roy, Rochester, N.Y., USA) in the dual wave length mode of operation with continuous measurements over 1 min every 1.5 min. After 45 min, test compounds were added and the reaction was continued for 45 min. After 45 min. A millimolar extinction coefficient of 19.1 cm⁻¹ was used for reduced ferricytochrome c. The results of the test compounds are provided in Table I. Extracts were made of the compounds in water unless otherwise indicated.

Table I provides the results of some arNOX inhibition experiments.

TABLE 1

| | | | | INHIBITION (−) |
|---|---|---|---|---|
| | | | ArNOX ACTIVITY | or |
| | | | % OF NO | STIMULATION |
| SAMPLE | SOLVENT | CONCENTRATION | ADDITION | (+) |
| Broccoli extract | Water | 25 μg/ml | 85 | −15 |
| (1.5%) | | | | |
| Shiitake (10%) | Water | 25 μg/ml | 82 | −18 |
| Coleus | Water | 25 μg/ml | 106 | +6 |
| Centella | Water | 25 μg/ml | +3 | +3 |
| asiatica | | | | |
| Lotus leaf | Water | 25 μg/ml | 98 | −2 |
| extract | | | | |
| Artichoke | Water | 25 μg/ml | 98 | −2 |
| (15%) | | | | |
| Sea rose | Water | 25 μg/ml | 96 | −4 |
| Tangerine | Water | 25 μg/ml | 94 | −6 |
| Oenothera | Water | 25 μg/ml | 94 | −6 |
| biennis seed | | | | |
| Natural | Ethanol | 25 μg/ml | 62 | −38 |
| astaxanthin | | | | |
| Red orange | Ethanol | 25 μg/ml | 98 | −2 |
| Schisandra | Water | 20/2 μg/ml | 0/84 | −100/16 |
| chinensis | 30% Ethanol | | 20/94 | 80/6 |
| | 70% Ethanol | | 77/97 | 23/3 |
| Lonicera | Water | 25 μg/ml | 20 | −81 |
| japonica | | | | |
| Rhizoma | Water | 25 μg/ml | 0 | −100 |
| Fagopyrum | 70% EtOH | | | |
| cymosum | | | | |
| Rhizoma | | 25 μg/ml | ~50% | ~−50% |
| Fagopyrum | | | | |
| dibotrys | | | | |
| β-Carotene | Water | 25 μg/ml | 28 | −72 |
| | Ethanol | 25 μg/ml | 68 | −32 |
| | Ethanol | 2.5 μg/ml | 50 | −50 |
| | Ethanol | 0.25 μg/ml | 73 | −42 |
| Narcissus | Water | 1/50 | 0 | −100 |
| tazetta (bulb | | | | |
| extract) | | | | |

### Example 2

### Topical Cosmetic Preparations

An eight-week controlled clinical usage study was conducted to screen six prototype anti-aging formulations containing plant extracts with arNOX-inhibiting properties for their efficacy and tolerability compared to a reference control. Efficacy was evaluated using clinical grading, bio-instrumentation measurements (Cutometer, Corneometer, Pro-Derm 2.0 imaging system, Chroma Meter), and self-assessment questionnaires. Tolerability was evaluated using irritation grading and monitoring for adverse events.

A total of 37 subjects completed study participation. Subjects qualified for study participation by having mild to moderate fine lines, coarse wrinkles, and hyperpigmentation on the right and left sides of the face. Subjects were assigned to one of the following test material groups according to a randomization design:
Control Product—No label (37 subjects)
Group 1 Product—Blue Label (Narcissus, Schizandra, Honeysuckle, Rhizoma Fagopyri, 6 subjects)
Group 2 Product—Yellow Label (Honeysuckle Extract, 6 subjects)
Group 3 Product—Red Label (Schizandra Extract, 7 subjects)
Group 4 Product—Green Label (Narcissus Extract, 5 subjects)
Group 5 Product—Yellow with Black Line Label (Fagopyrum Rhizoma Extract, 7 subjects)
Group 6 Product—Red with Black Line Label (Narcissus+Schizandra Extract, 6 subjects)
   Subjects were instructed to apply the assigned Group # product to the right or left side of the face and to apply Control Product—No Color Label to the opposite side of the face twice daily (in the morning and evening) after cleansing their faces.

Clinic evaluations were conducted at Baseline (Visit 1), Week 4 (Visit 2), and Week 8 (Visit 3). Subjects participated in the following clinical grading and instrumental procedures at each visit (unless otherwise indicated).

Efficacy/Performance Parameters

Subjects were clinically graded on the right and left sides of the face for the following parameters: fine wrinkles (periocular), coarse wrinkles (periocular), skin texture (cheeks), overall discoloration, brightness (cheeks), clarity of skin, pore size (forehead and nose area), pore distribution/structure, and overall skin radiance.

Irritation/Safety Parameter Grading

Subjects were clinically graded on the right and left sides of the face for objective irritation parameters (erythema, edema, scaling) and subjective irritation parameters (burning, stinging, itching, tightness, tingling).

Skin Surface Hydration Measurements

Skin surface hydration measurements were taken using the Corneometer® CM 825 (Courage+Khazaka, Germany) hydration analyzer. Measurements were taken (in triplicate) on the lower center of the left and right cheeks in order to quantify the moisture content of the stratum corneum.

Skin Luminance Measurements

Skin luminance measurements were made in triplicate using a Chroma Meter CR400 (Konica-Minolta, Japan) skin luminance analyzer and were taken on pigmented lesions (selected by the investigator) on the right and left sides of the face to instrumentally assess changes in skin color/tone.

Skin Viscoelasticity Measurements

A single viscoelasticity measurement was taken using the Cutometer® SEM 575 (Courage+Khazaka, Germany) viscoelasticity meter. Measurements were taken on the center of each subject's right and left cheeks in order to assess the viscoelastic properties of the skin.

Questionnaires

Subjects completed the following questionnaires at Week 4 and Week 8.
Subject Skin Change Evaluation questionnaire regarding changes in skin condition parameters since the start of the study
Subject Evaluation questionnaire regarding the current condition of skin condition parameters and test material attributes and tolerance

After eight weeks of product use, the Control, Group 3 and Group 5 showed significant improvements in ten of the eleven grading parameters, while Groups 1 and 6 showed significant improvements in nine of the eleven grading parameters (excluding pore distribution and clarity, respectively). None of the groups showed an improvement in periocular coarse wrinkles. Group 4 showed improvements in four grading parameters (fine wrinkles, tactile roughness, brightness and overall radiance).

Clinical grading for erythema and skin luminance (Chroma Meter CR400) results showed that the Control, Groups 3 and 6 performed the best in reducing facial redness. Improvements in this parameter were observed by the clinical grader (but not Chroma Meter) for Group 5. Skin Hydration (Corneometer® CM 825) results showed that improvements in miniaturization were observed at both visits for Control and Group 4 (Groups 3 and 6 showed improvements at Week 4 that did not persist to Week 8).

Attrition

Thirty-seven (37) subjects completed the study. Forty-three (43) subjects enrolled for study participation and six (6) subjects were discontinued due to the following reasons:
Voluntarily discontinued/scheduling conflict: 020, 022, 029
Failure to attend scheduled visit: 009, 034
Investigator discretion: 010

Adverse Events

There were no adverse events reported by subjects during the course of the study.

Subject Demographics

Thirty-seven (37) female subjects completed the study. Following is a summary of the demographic information (age, ethnicity, and Fitzpatrick Skin Classification) for all subjects. For ethnicity and Fitzpatrick type, the number of subjects in each category is listed with the percentage of the subject population in parentheses. Ethnicity information was obtained from each subject's Eligibility and Health Questionnaire. Table II provides the demographic information for the study subjects.

TABLE II
Summary Of Demographic Information
Demographic Summary

| | | |
|---|---|---|
| Age | Mean Age ± Standard | 53.90 ± 6.02 |
| (Years) | Deviation | |
| | Minimum Age | 42.54 |
| | Maximum Age | 66.23 |
| Ethnicity | Asian | 2 (5.4%) |
| | Caucasian | 34 (91.9%) |
| | Hispanic | 1 (2.7%) |
| Fitzpatrick Skin | Type I | 4 (10.8%) |
| Classification | Type II | 23 (62.2%) |
| | Type III | 10 (27.0%) |

The Fitzpatrick Skin Classification is based on the skin's unprotected response to the first 30-45 minutes of sun exposure after a winter season without sun exposure. The categories of the skin types are as follows:
I Always burns easily; never tans.
II Always burns easily; tans minimally
III Burns moderately; tans gradually
IV Burns minimally; always tans well
V Rarely burns; tans profusely
VI Never burns; deeply pigmented

### Example 3

### Procedures and Methods

Prior to the start of the study, prospective subjects participated in a three-day washout period, during which facial moisturizers were not applied to the face.

At Baseline (Visit 1), prospective subjects washed their faces and removed all make-up at least 30 minutes prior to arriving at the clinic. Prospective subjects brought their regular skin care regimen products for eligibility consideration. Subjects completed an Eligibility and Health Questionnaire and signed an Informed Consent Agreement, a Confidentiality Agreement, and a Photography Release Form.

Subjects participated in the following clinical grading procedures:

Efficacy/Performance Parameters

Subjects were clinically graded on the right and left sides of the face for the following parameters:
Fine Wrinkles—periocular area
Coarse Wrinkles—periocular area
Skin Texture (Visual Appearance)—cheeks
Tactile Roughness—cheeks
Overall Discoloration
Brightness (Shine/Reflection)—cheeks
Clarity of Skin (No Marks/Blemishes)
Pore Size—forehead and nose area
Pore Distribution/Structure (Evenness)
Overall Skin Radiance

Results of the efficacy/performance parameter grading were recorded using the following 1 to 10 point scale:
1=Positive (1 to 3=Good/Desirable)
10=Negative (8 to 10=Undesirable)
Half-point scores were used as needed

Subjects qualified for continued study participation by having a score of 2 to 7 for periocular fine lines and hyperpigmentation, and a score of 2 to 5 for periocular coarse wrinkles.

Irritation/Safety Parameter Grading

Subjects were clinically graded on the right and left sides of the face for objective irritation parameters (erythema, edema, scaling) and subjective irritation parameters (burning, stinging, itching, tightness, tingling). Results of the irritation grading were recorded using the following scale:
0=None
1=Mild
2=Moderate
3=Severe
Half-points were used as necessary
   Qualified subjects participated in the following instrumentation measurements:

### Example 4

### Skin Surface Hydration Measurements

Skin surface hydration measurements were taken using the Corneometer® CM 825 (Courage+Khazaka, Germany) hydration analyzer. Measurements were made in triplicate and were taken on the lower center of the left and right cheeks in order to quantify the moisture content of the stratum corneum. The measuring principle of the Corneometer® is based on capacitance measurement of a dielectric medium. Any change in the dielectric constant due to skin surface hydration variation alters the capacitance of a precision measuring capacitor. These measurements can detect very slightest changes in the hydration level of the skin with very high reproducibility. Readings are directly proportional to the skin's electrical capacitance and measurements increase as the skin becomes more hydrated.

### Example 5

### Skin Luminance Measurements

Skin luminance measurements were made in triplicate using a Chroma Meter CR400 (Konica-Minolta, Japan) skin luminance analyzer and were taken on pigmented lesions (selected by the investigator) on the right and left sides of the face. The Chroma Meter instrumentally (and objectively) assesses changes in skin color/tone. An additional Chroma Meter measurement was taken on a non-pigmented (normal) area on one side of the face. The Chroma Meter is a sensitive calorimeter that allows the setting and calibration of color-difference target colors. The Chroma Meter has a detachable head for easy and independent analysis of selected areas. The following values were recorded:
L*: Describes the relative brightness on a gray scale from black to white; values increase as the skin becomes brighter and lighter
a*: Describes the color hue ranging from red to green; values increase with improvements in skin vascularization, increased blood flow, and improved skin tone
b*: Describes the color hue ranging from blue to yellow; values typically decrease with skin lightening
   An additional Chroma Meter measurement was taken on a non-pigmented (normal) area on one side of the face for each subject.

### Example 6

### Skin Visco-Elasticity Measurements

A single visco-elasticity measurement was taken using the Cutometer® SEM 575 (Courage+Khazaka, Germany) viscoelasticity meter. Measurements were taken on the center of each subject's right and left cheeks in order to assess the viscoelastic properties of the skin. The measuring principle is based on suction. Negative pressure is created in the device and the skin is drawn into the aperture of the probe. Inside the probe, the penetration depth is determined by a non-contact optical measuring system. The light intensity varies due to the penetration depth of the skin. The resistance of the skin to be sucked up by the negative pressure (firmness and its ability to return into its original position (elasticity) are displayed on the instrument as curves at the end of each measurement. Three-hundred (300) mbar of negative pressure was applied and released through an 8-millimeter (mm) probe. The movement of the skin into and out of the probe was recorded during the application and release of suction, and resiliency and extensibility were calculated.

Subjects were assigned to one of the following test material groups according to a randomization design:
Control Product—No label (37 subjects)
Group 1 Product—Blue Label (6 subjects)
Group 2 Product—Yellow Label (6 subjects)
Group 3 Product—Red Label (7 subjects)
Group 4 Product—Green Label (5 subjects)
Group 5 Product—Yellow with Black Line Label (7 subjects)
Group 6 Product—Red with Black Line Label (6 subjects)

Subjects were instructed to apply the assigned Group # product to the right or left side of the face and to apply Control Product—No Color Label to the opposite side of the face (as determined by a randomization design) according to the following usage instructions.

Apply a thin layer twice daily in the morning and evening after cleansing your face. Moisturizers and makeup products may be applied after.

The formulations for each of the compositions are provided below in Table 3.

TABLE 3

| arNOX - Control Gel | n = 37 | |
|---|---|---|
| Quantitative Product Formulation | | |
| Lab Formula Number: AB-87-04A | | |
| INCI | W/W % | **Supplier |
| Water (Aqua) | 98.980000 | House |
| Acrylates/C10-31 Alkyl Acrylate | 0.300000 | Noveon |
| Crosspolyme | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Total: | 100.000000 | |
| Group 1: Blue Label | n = 6 | |
| arNOX - Combo Extract Formulation | | |
| Quantitative Product Formulation | | |
| Lab Formula Number: AB-87-06B | | |
| INCI | W/W % | **Supplier |
| Water (Aqua) | 63.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Water (Aqua) | 5.700000 | House |
| Glycerin | 13.300000 | House |
| Water (Aqua) | 0.900000 | House |
| Narcissus tazetta Bulb Extract | 0.100000 | Symrise |
| Water (Aqua) | 1.492500 | House |
| Glycerin | 3.482500 | House |
| Schizandra chinenesis Fruit/Seed | 0.025000 | Draco |
| Extract* | | |
| Water (Aqua) | 1.492500 | House |
| Glycerin | 3.482500 | House |
| Lonicera caprifolium (Honeysuckle) | 0.025000 | Phytoway |
| Extract* | | |
| Water (Aqua) | 1.492500 | House |
| Glycerin | 3.482500 | House |
| Rhizoma Fagopyri dibotrys Extract* | 0.025000 | Xuancheng |
| | | Baicao |
| Total: | 100.000000 | |
| Group 2: Yellow Label | n = 6 | |
| arNOX - Honeysuckle Extract | | |
| Formulation | | |
| Quantitative Product Formulation | | |
| Lab Formula Number: AB-87-03B | | |
| INCI | W/W % | **Supplier |
| Water (Aqua) | 78.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Water (Aqua) | 5.970000 | House |
| Glycerin | 13.930000 | House |
| Lonicera caprifolium (Honeysuckle) | 0.100000 | Phytoway |
| Extract* | | |
| Total: | 100.000000 | |
| Group 3: Red Label | n = 6 | |
| arNOX - Schizandra Extract | | |
| Formulation | | |
| Quantitative Product Formulation | | |
| Lab Formula Number: AB-87-03A | | |
| INCI | W/W % | **Supplier |
| Water (Aqua) | 78.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Water (Aqua) | 5.970000 | House |
| Glycerin | 13.930000 | House |
| Schizandra chinenesis Fruit/Seed | 0.100000 | Draco |
| Extract* | | |
| Total: | 100.000000 | |
| Group 4: Green Label | n = 5 | |
| arNOX - Narcissus Extract | | |
| Formulation | | |
| Quantitative Product Formulation | | |
| Lab Formula Number: AB-87-06A | | |
| INCI | W/W % | **Supplier |
| Water (Aqua) | 78.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Water (Aqua) | 5.700000 | House |
| Glycerin | 13.300000 | House |
| Water (Aqua) | 0.900000 | House |
| Narcissus tazetta Bulb Extract | 0.100000 | Symrise |
| Total: | 100.000000 | |
| Group 5: Yellow/black Label | n = 7 | |
| arNOX - Rhizoma Fagopyri Extract | | |
| Formulation | | |
| Quantitative Product Formulation | | |
| Lab Formula Number: AB-87-03C | | |
| INCI | W/W % | **Supplier |
| Water (Aqua) | 78.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Water (Aqua) | 5.970000 | House |
| Glycerin | 13.930000 | House |
| Rhizoma Fagopyri dibotrys Extract* | 0.100000 | Xuancheng |
| | | Baico |
| Total: | 100.000000 | |
| Group 6: Red/black Label | n = 6 | |
| arNOX - Narcissus + Schizandra Extract | | |
| Formulation | | |
| Quantitative Product Formulation | | |
| Lab Formula Number: AB-87-06C | | |
| INCI | W/W % | **Supplier |
| Water (Aqua) | 68.980000 | House |
| Acrylates/C10-31 Alkyl Acrylate | 0.300000 | Noveon |
| Crosspolymer | | |
| Methylparaben | 0.150000 | Clariant |
| Chlorphenesin | 0.300000 | House |
| Aminomethyl Propanol | 0.150000 | Angus |
| Polysorbate 20 | 0.100000 | Unigema |
| Fragrance (Parfum) | 0.020000 | Ungerer |
| Water (Aqua) | 5.700000 | House |
| Glycerin | 13.300000 | House |
| Water (Aqua) | 0.900000 | House |
| Narcissus tazetta Bulb Extract | 0.100000 | Symrise |
| Water (Aqua) | 2.985000 | House |
| Glycerin | 6.965000 | House |
| Schizandra chinenesis Fruit/Seed | 0.050000 | Draco |
| Extract* | | |
| Total: | 100.000000 | |

**Noveon IP Holdings Corp. Cleveland, Ohio, U.S. Clariant, Corp. Charlotte, N.C., U.S. Angus Chemical Co., Buffalo Grove Il, U.S. Unigema, New Castle, DE, U.S. Symrise Inc., Teterboro, NJ Draco Natural Products, Inc., San Jose, CA, U.S.A. Xuancheng Baicao Plants Industry and Trade CO., LTD, Anhui, China

Subjects were provided with written usage instructions, a calendar of future visits, and a daily diary to record test material application times and comments.

Subjects returned to the clinic at Week 4 (Visit 2) and Week 8 (Visit 3). Subjects washed their faces and removed makeup at least 30 minutes prior to coming to the test facility for each visit. Subjects also brought their test materials to each visit for usage compliance checks. Subjects participated in the following procedures at each visit:
Efficacy/performance parameter grading
Irritation/safety parameter grading
Skin Surface Hydration (Corneometer®) measurements
Skin Luminence (Chroma Meter) measurements
Skin Visco-elasticity (Cutometer®) measurements
   Subjects also completed a Subject Skin Change Evaluation Questionnaire and a Subject Evaluation Questionnaire regarding test material attributes, tolerance, and improvements in skin condition parameters on the right and left sides of the face.

Daily diaries were returned to the clinic at each visit, and new diaries were distributed at Visits 2. Subjects returned test material units to the clinic at the completion of the study. Daily diaries were reviewed by clinic personnel and test material units were weighed at each visit to ensure compliance.

### Example 7

### Biostatistics and Data Management

Mean values for clinical grading parameters and instrumentation measurements at Week 4 and Week 8 were statistically compared to mean Baseline values using a paired t-test at the p≦0.05 significance level. Mean percent change from Baseline and incidence of improvement were calculated for all attributes. Comparisons were made among the seven test materials using analysis of variance (ANOVA) with paired comparisons (Fisher's LSD). See Appendix I for complete statistical calculations.

### Example 8

### Results

At Baseline, Week 4, and Week 8, subjects had the following clinical grading and instrumentation procedures performed on the right and left sides of the face:
Efficacy/performance parameter grading;
Irritation/safety parameter grading;
Corneometer measurements to assess miniaturization;
Chroma Meter measurements to instrumentally assess changes in skin color/tone taken on pigmented lesions;
Cutometer measurements to assess the visco-elastic properties of the skin.

Table 4 (on the following pages) presents the results of the clinical grading and instrumentation for each test material. Mean values at Week 4 and Week 8 are statistically compared to mean Baseline values for significant differences. The average percent change from Baseline is listed in parentheses (—indicates this value could not be calculated).

TABLE 4
MEAN VALUES FOR CLINICAL GRADING AND INSTRUMENTATION
PROCEDURES

| | | Control Product - No Color Label (n = 37) | | | | | | |
|---|---|---|---|---|---|---|---|---|
| | | Baseline | Week 4 | | | Week 8 | | |
| | | (Visit 1) | (Visit 3) | | | (Visit 4) | | |
| EFFICACY/PERFORMANCE GRADING | | | | | | | | |
| Fine Wrinkles - periocular area | | 4.97 | 4.92 | | (−1.0%) | 4.85 | [symbol] | (−2.4%) |
| Coarse Wrinkles - periocular area | | 3.68 | 3.68 | | (0.0%) | 3.65 | | (−0.7%) |
| Skin Texture (Visual Appearance) - cheeks | | 5.42 | 5.23 | [symbol] | (−3.4%) | 4.97 | [symbol] | (−8.2%) |
| Tactile Roughness - cheeks | | 4.41 | 3.86 | [symbol] | (−12.2%) | 3.45 | [symbol] | (−21.7%) |
| Overall Discoloration | | 5.04 | 4.97 | | (−1.3%) | 4.82 | [symbol] | (−4.2%) |
| Brightness (Shine/Reflection) - cheeks | | 5.41 | 5.07 | [symbol] | (−6.2%) | 4.69 | [symbol] | (−13.2%) |
| Clarity of Skin (No Marks/Blemishes) | | 5.00 | 4.66 | [symbol] | (−6.7%) | 4.57 | [symbol] | (−8.6%) |
| Pore Size - forehead | | 4.19 | 3.97 | [symbol] | (−5.1%) | 3.77 | [symbol] | (−10.0%) |
| Pore Size - nose area | | 4.74 | 4.54 | [symbol] | (−4.2%) | 4.32 | [symbol] | (−8.8%) |
| Pore Distribution/Structure (Evenness) | | 4.51 | 4.31 | [symbol] | (−4.4%) | 4.15 | [symbol] | (−8.0%) |
| Overall Skin Radiance | | 5.39 | 5.18 | [symbol] | (−4.0%) | 4.91 | [symbol] | (−9.0%) |
| IRRITATION/SAFETY GRADING | | | | | | | | |
| Erythema | | 0.42 | 0.12 | [symbol] | (−70.9%) | 0.11 | [symbol] | (−74.1%) |
| Edema | | 0.00 | 0.00 | | — | 0.00 | | — |
| Scaling | | 0.01 | 0.00 | | (−100.0%) | 0.00 | | (−100.0%) |
| Burning | | 0.00 | 0.00 | | — | 0.00 | | — |
| Stinging | | 0.00 | 0.00 | | — | 0.00 | | — |
| Itching | | 0.00 | 0.00 | | — | 0.00 | | — |
| Tightness | | 0.15 | 0.00 | [symbol] | (−100.0%) | 0.00 | [symbol] | (−100.0%) |
| Tingling | | 0.00 | 0.00 | | — | 0.00 | | — |
| CORNEOMETER MEASUREMENTS | | 50.92 | 59.23 | [symbol] | (16.3%) | 57.17 | [symbol] | (12.2%) |
| CHROMA METER MEASUREMENTS | | | | | | | | |
| Pigmented Lesion | L* | 60.68 | 60.57 | | (−0.1%) | 60.06 | | (−1.0%) |
| | a* | 13.40 | 12.27 | [symbol] | (−8.3%) | 12.79 | [symbol] | (−4.5%) |
| | b* | 15.74 | 15.96 | | (1.4%) | 15.93 | | (1.2%) |

| | | | | | |
|---|---|---|---|---|---|
| CUTOMETER MEASUREMENTS | | | | | |
| Biological Elasticity | 0.37 | 0.38 | (0.6%) | 0.38 | (2.6%) |
| Extensibility | 1.15 | 1.16 | (1.2%) | 1.25 | (8.6%) |
| Pure Elasticity | 0.54 | 0.55 | (1.5%) | 0.56 | (2.0%) |
| Resiliency | 0.71 | 0.71 | (0.0%) | 0.73 | (2.7%) |

arNOX - Combo Extract Formulation

| | | Group 1 Product - Blue Label (n = 6) | | | | | | |
|---|---|---|---|---|---|---|---|---|
| | | Baseline | Week 4 | | | Week 8 | | |
| | | (Visit 1) | (Visit 3) | | | (Visit 4) | | |
| EFFICACY/PERFORMANCE GRADING | | | | | | | | |
| Fine Wrinkles - periocular area | | 5.17 | 4.67 | [symbol] | (−9.6%) | 4.17 | [symbol] | (−19.3%) |
| Coarse Wrinkles - periocular area | | 3.50 | 3.50 | | (0.0%) | 3.33 | | (−4.7%) |
| Skin Texture (Visual Appearance) - cheeks | | 5.50 | 5.00 | | (−9.0%) | 4.33 | [symbol] | (−21.2%) |
| Tactile Roughness - cheeks | | 4.58 | 3.75 | [symbol] | (−18.1%) | 2.50 | [symbol] | (−45.4%) |
| Overall Discoloration | | 5.08 | 4.58 | [symbol] | (−9.8%) | 4.25 | [symbol] | (−16.3%) |
| Brightness (Shine/Reflection) - cheeks | | 5.58 | 4.83 | [symbol] | (−13.4%) | 4.08 | [symbol] | (−26.8%) |
| Clarity of Skin (No Marks/Blemishes) | | 4.92 | 4.33 | [symbol] | (−11.8%) | 3.75 | [symbol] | (−23.7%) |
| Pore Size - forehead | | 4.25 | 3.75 | | (−11.7%) | 3.25 | [symbol] | (−23.5%) |
| Pore Size - nose area | | 4.92 | 4.42 | | (−10.1%) | 3.75 | [symbol] | (−23.7%) |
| Pore Distribution/Structure (Evenness) | | 4.50 | 4.08 | | (−9.2%) | 3.67 | | (−18.5%) |
| Overall Skin Radiance | | 5.50 | 4.75 | [symbol] | (−13.6%) | 4.00 | [symbol] | (−27.2%) |
| IRRITATION/SAFETY GRADING | | | | | | | | |
| Erythema | | 0.33 | 0.08 | | (−75.0%) | 0.33 | | (0.0%) |
| Edema | | 0.00 | 0.00 | | — | 0.00 | | — |
| Scaling | | 0.00 | 0.00 | | — | 0.00 | | — |
| Burning | | 0.00 | 0.00 | | — | 0.00 | | — |
| Stinging | | 0.00 | 0.00 | | — | 0.00 | | — |
| Itching | | 0.00 | 0.00 | | — | 0.00 | | — |
| Tightness | | 0.17 | 0.00 | | (−100.0%) | 0.00 | | (−100.0%) |
| Tingling | | 0.00 | 0.00 | | — | 0.00 | | — |
| CORNEOMETER MEASUREMENTS | | 51.67 | 66.11 | | (27.9%) | 51.72 | | (0.1%) |
| CHROMA METER MEASUREMENTS | | | | | | | | |
| Pigmented Lesion | L* | 59.78 | 58.19 | | (−2.6%) | 56.26 | [symbol] | (−5.8%) |
| | a* | 12.58 | 12.84 | | (2.0%) | 13.46 | | (7.0%) |
| | b* | 15.55 | 16.14 | | (3.7%) | 16.24 | [symbol] | (4.4%) |

| | | | | | | |
|---|---|---|---|---|---|---|
| CUTOMETER MEASUREMENTS | | | | | | |
| Biological Elasticity | 0.33 | 0.36 | | (7.0%) | 0.40 | (19.2%) |
| Extensibility | 1.39 | 1.29 | | (−7.1%) | 1.16 | (−16.6%) |
| Pure Elasticity | 0.48 | 0.55 | [symbol] | (14.9%) | 0.59 | (22.8%) |
| Resiliency | 0.63 | 0.65 | | (3.3%) | 0.74 | (16.6%) |

arNOX - Honeysuckle Extract Formulation

| | | Group 2 Product - Yellow Label (n = 6) | | | | | | |
|---|---|---|---|---|---|---|---|---|
| | | Baseline | Week 4 | | | Week 8 | | |
| | | (Visit 1) | (Visit 3) | | | (Visit 4) | | |
| EFFICACY/PERFORMANCE GRADING | | | | | | | | |
| Fine Wrinkles - periocular area | | 4.58 | 3.92 | [symbol] | (−14.5%) | 3.42 | [symbol] | (−25.4%) |
| Coarse Wrinkles - periocular area | | 3.33 | 3.33 | | (0.0%) | 3.17 | | (−5.0%) |
| Skin Texture (Visual Appearance) - cheeks | | 5.42 | 4.92 | | (−9.2%) | 4.33 | [symbol] | (−20.0%) |
| Tactile Roughness - cheeks | | 4.67 | 3.67 | [symbol] | (−21.4%) | 3.17 | [symbol] | (−32.1%) |
| Overall Discoloration | | 5.50 | 4.58 | [symbol] | (−16.6%) | 4.33 | [symbol] | (−21.2%) |
| Brightness (Shine/Reflection) - cheeks | | 5.33 | 4.58 | [symbol] | (−14.0%) | 4.00 | [symbol] | (−25.0%) |
| Clarity of Skin (No Marks/Blemishes) | | 5.00 | 4.33 | [symbol] | (−13.3%) | 4.00 | [symbol] | (−20.0%) |
| Pore Size - forehead | | 4.08 | 3.33 | [symbol] | (−18.3%) | 2.83 | [symbol] | (−30.6%) |
| Pore Size - nose area | | 4.58 | 3.75 | [symbol] | (−18.1%) | 3.25 | [symbol] | (−29.0%) |
| Pore Distribution/Structure (Evenness) | | 4.67 | 3.92 | [symbol] | (−16.0%) | 3.42 | [symbol] | (−26.7%) |
| Overall Skin Radiance | | 5.17 | 4.67 | [symbol] | (−9.6%) | 3.83 | [symbol] | (−25.8%) |
| IRRITATION/SAFETY GRADING | | | | | | | | |
| Erythema | | 0.33 | 0.17 | | (−50.0%) | 0.17 | | (−50.0%) |
| Edema | | 0.00 | 0.00 | | — | 0.00 | | — |
| Scaling | | 0.00 | 0.00 | | — | 0.00 | | — |
| Burning | | 0.00 | 0.00 | | — | 0.00 | | — |
| Stinging | | 0.00 | 0.00 | | — | 0.00 | | — |
| Itching | | 0.00 | 0.00 | | — | 0.00 | | — |
| Tightness | | 0.08 | 0.00 | | (−100.0%) | 0.00 | | (−100.0%) |
| Tingling | | 0.00 | 0.00 | | — | 0.00 | | — |
| CORNEOMETER MEASUREMENTS | | 55.28 | 62.33 | | (12.7%) | 58.89 | | (6.5%) |
| CHROMA METER MEASUREMENTS | | | | | | | | |
| Pigmented Lesion | L* | 60.29 | 61.12 | | (1.3%) | 60.20 | | (−0.1%) |
| | a* | 13.75 | 11.15 | [symbol] | (−18.9%) | 12.68 | | (−7.7%) |
| | b* | 14.60 | 15.69 | | (7.5%) | 15.25 | | (4.5%) |

| | | | | | | |
|---|---|---|---|---|---|---|
| CUTOMETER MEASUREMENTS | | | | | | |
| Biological Elasticity | 0.36 | 0.37 | (1.7%) | 0.37 | | (1.0%) |
| Extensibility | 1.14 | 1.17 | (2.8%) | 1.30 | * | (14.4%) |
| Pure Elasticity | 0.54 | 0.58 | (9.1%) | 0.59 | | (9.9%) |
| Resiliency | 0.68 | 0.68 | (0.9%) | 0.68 | | (0.8%) |

arNOX - Schizandra Extract Formulation

| | | Group 3 Product - Red Label (n = 7) | | | | | | |
|---|---|---|---|---|---|---|---|---|
| | | Baseline | Week 4 | | | Week 8 | | |
| | | (Visit 1) | (Visit 3) | | | (Visit 4) | | |
| EFFICACY/PERFORMANCE GRADING | | | | | | | | |
| Fine Wrinkles - periocular area | | 5.93 | 5.43 | | (−8.4%) | 5.00 | [symbol] | (−15.6%) |
| Coarse Wrinkles - periocular area | | 4.00 | 4.00 | | (0.0%) | 3.93 | | (−1.7%) |
| Skin Texture (Visual Appearance) - cheeks | | 5.36 | 4.86 | | (−9.3%) | 4.21 | [symbol] | (−21.3%) |
| Tactile Roughness - cheeks | | 3.71 | 2.93 | [symbol] | (−21.1%) | 2.29 | [symbol] | (−38.4%) |
| Overall Discoloration | | 5.57 | 5.57 | | (0.0%) | 4.71 | [symbol] | (−15.3%) |
| Brightness (Shine/Reflection) - cheeks | | 5.50 | 4.79 | [symbol] | (−12.9%) | 4.00 | [symbol] | (−27.2%) |
| Clarity of Skin (No Marks/Blemishes) | | 5.29 | 4.43 | [symbol] | (−16.2%) | 4.14 | [symbol] | (−21.6%) |
| Pore Size - forehead | | 4.07 | 3.43 | [symbol] | (−15.7%) | 2.93 | [symbol] | (−28.0%) |
| Pore Size - nose area | | 4.29 | 3.57 | [symbol] | (−16.6%) | 3.14 | [symbol] | (−26.6%) |
| Pore Distribution/Structure (Evenness) | | 4.29 | 3.79 | [symbol] | (−11.6%) | 3.50 | [symbol] | (−18.3%) |
| Overall Skin Radiance | | 5.21 | 4.71 | [symbol] | (−9.5%) | 4.21 | [symbol] | (−19.1%) |
| IRRITATION/SAFETY GRADING | | | | | | | | |
| Erythema | | 0.71 | 0.14 | [symbol] | (−80.0%) | 0.07 | [symbol] | (−90.0%) |
| Edema | | 0.00 | 0.00 | | — | 0.00 | | — |
| Scaling | | 0.21 | 0.00 | | (−100.0%) | 0.00 | | (−100.0%) |
| Burning | | 0.00 | 0.00 | | — | 0.00 | | — |
| Stinging | | 0.00 | 0.00 | | — | 0.00 | | — |
| Itching | | 0.00 | 0.00 | | — | 0.00 | | — |
| Tightness | | 0.14 | 0.00 | | (−100.0%) | 0.00 | | (−100.0%) |
| Tingling | | 0.00 | 0.00 | | — | 0.00 | | — |
| CORNEOMETER MEASUREMENTS | | 42.10 | 66.43 | [symbol] | (57.8%) | 55.52 | | (31.9%) |
| CHROMA METER MEASUREMENTS | | | | | | | | |
| Pigmented Lesion | L* | 60.07 | 59.66 | | (−0.6%) | 59.63 | | (−0.7%) |
| | a* | 13.59 | 12.12 | | (−10.8%) | 12.15 | [symbol] | (−10.6%) |
| | b* | 16.69 | 16.29 | | (−2.4%) | 16.22 | | (−2.8%) |

| | | | | | | | |
|---|---|---|---|---|---|---|---|
| CUTOMETER MEASUREMENTS | | | | | | | |
| Biological Elasticity | 0.35 | 0.40 | [symbol] | (14.2%) | 0.38 | [symbol] | (10.7%) |
| Extensibility | 1.03 | 1.09 | | (5.9%) | 1.16 | * | (12.6%) |
| Pure Elasticity | 0.52 | 0.59 | [symbol] | (14.4%) | 0.58 | * | (12.0%) |
| Resiliency | 0.66 | 0.73 | [symbol] | (10.3%) | 0.72 | [symbol] | (8.3%) |

arNOX - Narcissus Extract Formulation

| | | Group 4 Product - Green Label (n = 5) | | | | | | |
|---|---|---|---|---|---|---|---|---|
| | | Baseline | Week 4 | | | Week 8 | | |
| | | (Visit 1) | (Visit 3) | | | (Visit 4) | | |
| EFFICACY/PERFORMANCE GRADING | | | | | | | | |
| Fine Wrinkles - periocular area | | 4.40 | 4.10 | | (−6.8%) | 3.60 | [symbol] | (−18.1%) |
| Coarse Wrinkles - periocular area | | 3.40 | 3.40 | | (0.0%) | 3.40 | | (0.0%) |
| Skin Texture (Visual Appearance) - cheeks | | 5.60 | 5.30 | | (−5.3%) | 4.70 | | (−16.0%) |
| Tactile Roughness - cheeks | | 4.80 | 4.10 | [symbol] | (−14.5%) | 3.50 | [symbol] | (−27.0%) |
| Overall Discoloration | | 4.60 | 4.40 | | (−4.3%) | 4.00 | | (−13.0%) |
| Brightness (Shine/Reflection) - cheeks | | 5.50 | 5.10 | | (−7.2%) | 4.00 | [symbol] | (−27.2%) |
| Clarity of Skin (No Marks/Blemishes) | | 5.20 | 5.00 | | (−3.8%) | 4.10 | | (−21.1%) |
| Pore Size - forehead | | 4.40 | 4.00 | | (−9.0%) | 3.50 | | (−20.4%) |
| Pore Size - nose area | | 5.10 | 4.90 | | (−3.9%) | 4.70 | | (−7.8%) |
| Pore Distribution/Structure (Evenness) | | 4.80 | 4.60 | | (−4.1%) | 4.10 | | (−14.5%) |
| Overall Skin Radiance | | 5.70 | 5.20 | | (−8.7%) | 4.40 | [symbol] | (−22.8%) |
| IRRITATION/SAFETY GRADING | | | | | | | | |
| Erythema | | 0.30 | 0.00 | | (−100.0%) | 0.00 | | (−100.0%) |
| Edema | | 0.00 | 0.00 | | — | 0.00 | | — |
| Scaling | | 0.00 | 0.00 | | — | 0.00 | | — |
| Burning | | 0.00 | 0.00 | | — | 0.00 | | — |
| Stinging | | 0.00 | 0.00 | | — | 0.00 | | — |
| Itching | | 0.00 | 0.00 | | — | 0.00 | | — |
| Tightness | | 0.10 | 0.00 | | (−100.0%) | 0.00 | | (−100.0%) |
| Tingling | | 0.00 | 0.10 | | — | 0.00 | | — |
| CORNEOMETER MEASUREMENTS | | 36.80 | 70.73 | [symbol] | (92.2%) | 56.87 | [symbol] | (54.5%) |
| CHROMA METER MEASUREMENTS | | | | | | | | |
| Pigmented Lesion | L* | 60.26 | 58.33 | | (−3.1%) | 60.39 | | (0.2%) |
| | a* | 14.23 | 13.67 | | (−3.9%) | 13.05 | | (−8.2%) |
| | b* | 15.16 | 15.72 | | (3.6%) | 15.54 | | (2.5%) |

| | | | | | | |
|---|---|---|---|---|---|---|
| CUTOMETER MEASUREMENTS | | | | | | |
| Biological Elasticity | 0.36 | 0.40 | | (9.2%) | 0.38 | (5.1%) |
| Extensibility | 1.15 | 1.23 | | (6.6%) | 1.19 | (3.8%) |
| Pure Elasticity | 0.55 | 0.59 | [symbol] | (7.2%) | 0.58 | (5.8%) |
| Resiliency | 0.71 | 0.76 | | (6.8%) | 0.70 | (−0.8%) |

arNOX - Rhizoma Fagopyri Extract
Formulation

| | | Group 5 Product - Yellow with Black Line Label (n = 7) | | | | | | |
|---|---|---|---|---|---|---|---|---|
| | | Baseline | Week 4 | | | Week 8 | | |
| | | (Visit 1) | (Visit 3) | | | (Visit 4) | | |
| EFFICACY/PERFORMANCE GRADING | | | | | | | | |
| Fine Wrinkles - periocular area | | 5.00 | 4.43 | | (−11.4%) | 4.00 | [symbol] | (−20.0%) |
| Coarse Wrinkles - periocular area | | 4.07 | 4.07 | | (0.0%) | 4.00 | | (−1.7%) |
| Skin Texture (Visual Appearance) - cheeks | | 5.07 | 4.57 | [symbol] | (−9.8%) | 4.00 | [symbol] | (−21.1%) |
| Tactile Roughness - cheeks | | 4.00 | 3.14 | [symbol] | (−21.4%) | 2.50 | [symbol] | (−37.5%) |
| Overall Discoloration | | 5.50 | 4.50 | [symbol] | (−18.1%) | 4.14 | [symbol] | (−24.6%) |
| Brightness (Shine/Reflection) - cheeks | | 5.21 | 4.43 | [symbol] | (−15.0%) | 4.07 | [symbol] | (−21.9%) |
| Clarity of Skin (No Marks/Blemishes) | | 5.07 | 4.29 | [symbol] | (−15.4%) | 3.93 | [symbol] | (−22.5%) |
| Pore Size - forehead | | 4.21 | 3.86 | | (−8.4%) | 3.36 | [symbol] | (−20.3%) |
| Pore Size - nose area | | 4.71 | 4.00 | [symbol] | (−15.1%) | 3.43 | [symbol] | (−27.2%) |
| Pore Distribution/Structure (Evenness) | | 4.71 | 4.07 | [symbol] | (−13.6%) | 3.57 | [symbol] | (−24.2%) |
| Overall Skin Radiance | | 5.79 | 5.00 | [symbol] | (−13.5%) | 4.29 | [symbol] | (−25.9%) |
| IRRITATION/SAFETY GRADING | | | | | | | | |
| Erythema | | 0.43 | 0.07 | [symbol] | (−83.3%) | 0.00 | [symbol] | (−100.0%) |
| Edema | | 0.00 | 0.00 | | — | 0.00 | | — |
| Scaling | | 0.00 | 0.00 | | — | 0.00 | | — |
| Burning | | 0.00 | 0.00 | | — | 0.00 | | — |
| Stinging | | 0.00 | 0.00 | | — | 0.00 | | — |
| Itching | | 0.00 | 0.00 | | — | 0.00 | | — |
| Tightness | | 0.14 | 0.00 | | (−100.0%) | 0.00 | | (−100.0%) |
| Tingling | | 0.00 | 0.00 | | — | 0.00 | | — |
| CORNEOMETER MEASUREMENTS | | 51.86 | 66.24 | | (27.7%) | 62.00 | | (19.5%) |
| CHROMA METER MEASUREMENTS | | | | | | | | |
| Pigmented Lesion | L* | 60.17 | 59.66 | | (−0.8%) | 60.51 | | (0.5%) |
| | a* | 13.48 | 12.55 | | (−6.9%) | 12.69 | | (−5.8%) |
| | b* | 15.74 | 16.43 | | (4.3%) | 15.56 | | (−1.1%) |

| | | | | | | | |
|---|---|---|---|---|---|---|---|
| CUTOMETER MEASUREMENTS | | | | | | | |
| Biological Elasticity | 0.39 | 0.43 | [symbol] | (9.8%) | 0.41 | * | (6.6%) |
| Extensibility | 1.27 | 1.18 | | (−6.8%) | 1.29 | | (1.6%) |
| Pure Elasticity | 0.56 | 0.62 | [symbol] | (10.6%) | 0.59 | | (5.6%) |
| Resiliency | 0.73 | 0.77 | [symbol] | (5.8%) | 0.77 | [symbol] | (6.2%) |

arNOX-Narcissus + Schizandra Extract
Formulation

| | | Group 6 Product - Red with Black Line Label (n = 6) | | | | | | |
|---|---|---|---|---|---|---|---|---|
| | | Baseline | Week 4 | | | Week 8 | | |
| | | (Visit 1) | (Visit 3) | | | (Visit 4) | | |
| EFFICACY/PERFORMANCE GRADING | | | | | | | | |
| Fine Wrinkles - periocular area | | 4.50 | 3.83 | [symbol] | (−14.8%) | 3.67 | [symbol] | (−18.5%) |
| Coarse Wrinkles - periocular area | | 3.50 | 3.50 | | (0.0%) | 3.50 | | (0.0%) |
| Skin Texture (Visual Appearance) - cheeks | | 5.25 | 4.58 | [symbol] | (−12.6%) | 3.75 | [symbol] | (−28.5%) |
| Tactile Roughness - cheeks | | 5.17 | 4.33 | [symbol] | (−16.1%) | 3.00 | [symbol] | (−41.9%) |
| Overall Discoloration | | 5.00 | 4.58 | | (−8.3%) | 4.00 | [symbol] | (−20.0%) |
| Brightness (Shine/Reflection) - cheeks | | 5.08 | 4.25 | [symbol] | (−16.3%) | 3.33 | [symbol] | (−34.4%) |
| Clarity of Skin (No Marks/Blemishes) | | 5.17 | 4.50 | | (−12.9%) | 3.75 | | (−27.4%) |
| Pore Size - forehead | | 4.17 | 3.58 | [symbol] | (−14.0%) | 3.17 | [symbol] | (−24.0%) |
| Pore Size - nose area | | 5.00 | 4.50 | | (−10.0%) | 3.83 | [symbol] | (−23.3%) |
| Pore Distribution/Structure (Evenness) | | 4.75 | 4.17 | [symbol] | (−12.2%) | 3.67 | [symbol] | (−22.8%) |
| Overall Skin Radiance | | 5.17 | 4.58 | [symbol] | (−11.2%) | 3.67 | [symbol] | (−29.0%) |
| IRRITATION/SAFETY GRADING | | | | | | | | |
| Erythema | | 0.42 | 0.00 | [symbol] | (−100.0%) | 0.00 | [symbol] | (−100.0%) |
| Edema | | 0.00 | 0.00 | | — | 0.00 | | — |
| Scaling | | 0.00 | 0.00 | | — | 0.00 | | — |
| Burning | | 0.00 | 0.00 | | — | 0.00 | | — |
| Stinging | | 0.00 | 0.00 | | — | 0.00 | | — |
| Itching | | 0.00 | 0.00 | | — | 0.00 | | — |
| Tightness | | 0.25 | 0.00 | | (−100.0%) | 0.00 | | (−100.0%) |
| Tingling | | 0.00 | 0.00 | | — | 0.00 | | — |
| CORNEOMETER MEASUREMENTS | | 48.28 | 67.06 | [symbol] | (38.8%) | 59.06 | | (22.3%) |
| CHROMA METER MEASUREMENTS | | | | | | | | |
| Pigmented Lesion | L* | 60.09 | 61.78 | | (2.8%) | 61.57 | | (2.4%) |
| | a* | 14.44 | 11.97 | [symbol] | (−17.0%) | 12.38 | [symbol] | (−14.2%) |
| | b* | 15.59 | 16.38 | | (5.0%) | 16.55 | | (6.1%) |

| | | | | | | |
|---|---|---|---|---|---|---|
| CUTOMETER MEASUREMENTS | | | | | | |
| Biological Elasticity | 0.39 | 0.40 | (0.7%) | 0.40 | | (0.7%) |
| Extensibility | 1.08 | 1.09 | (1.6%) | 1.22 | [symbol] | (13.4%) |
| Pure Elasticity | 0.59 | 0.61 | (3.3%) | 0.59 | | (0.1%) |
| Resiliency | 0.72 | 0.73 | (1.1%) | 0.73 | | (1.2%) |

[symbol]

Indicates a statistically significant (p ≦ 0.05) decrease compared to Baseline [symbol]
Indicates a statistically significant (p ≦ 0.05) increase compared to Baseline

Results of Summary Statistics for Chroma Meter Measurements for Non-Pigmented Area (All Subjects) are provided in Table 5.

TABLE 5

| | | Baseline | | Week 4 | | Week 8 | |
|---|---|---|---|---|---|---|---|
| | | (n = 24) | | (n = 25) | | (n = 28) | |
| | | | Standard | | Standard | | Standard |
| | | Mean | Deviation | Mean | Deviation | Mean | Deviation |
| Chroma | L* | 62.38 | 2.67 | 62.02 | 3.5 | 61.79 | 2.66 |
| Meter: | a* | 13.97 | 3.33 | 11.43 | 2.9 | 12.51 | 2.81 |
| Non- | b* | 13.93 | 2.04 | 14.67 | 2.83 | 14.44 | 2.29 |
| Pig- | | | | | | | |
| mented/ | | | | | | | |
| Neutral | | | | | | | |
| Area | | | | | | | |

### Example 9

### Results of ANOVA Comparisons for Clinical Grading and Instrumentation

Comparisons, based on the average change from Baseline, were made among the seven treatments using analysis of variance (ANOVA) with paired comparisons (Fisher's LSD). The rankings, provided in Table 6, below, illustrate the statistically significant (p≦0.05) differences among the test groups. Rankings are presented in order of greatest to least improvement and parameters with no significant differences are not listed. The average change from Baseline is listed beneath each test material.

TABLE 6

| | Group 2 | Group 6 | Group 5 | Group 1 | Group 3 | Group 4 | Control |
|---|---|---|---|---|---|---|---|
| Fine Wrinkles - Week 4 | −0.67 | −0.67 | −0.57 | −0.50 | −0.50 | −0.30 | −0.05 |
| (p = <0.0001) | | | | | | | |
| | Group 2 | Group 1 | Group 5 | Group 3 | Group 6 | Group 4 | Control |
| Fine Wrinkles - Week 8 | −1.17 | −1.00 | −1.00 | −0.93 | −0.83 | −0.80 | −0.12 |
| (p = <0.0001) | | | | | | | |
| | Group 6 | Group 1 | Group 2 | Group 3 | Group 5 | Group 4 | Control |
| Skin Texture - Week 4 | −0.67 | −0.50 | −0.50 | −0.50 | −0.50 | −0.30 | −0.19 |
| (p = 0.0138) | | | | | | | |
| | Group 6 | Group 1 | Group 3 | Group 2 | Group 5 | Group 4 | Control |
| Skin Texture - Week 8 | −1.50 | −1.17 | −1.14 | −1.08 | −1.07 | −0.90 | −0.45 |
| (p = 0.0001) | | | | | | | |
| | Group 6 | Group 1 | Group 2 | Group 5 | Group 3 | Group 4 | Control |
| Tactile Roughness - Week 8 | −2.17 | −2.08 | −1.50 | −1.50 | −1.43 | −1.30 | −0.96 |
| (p = 0.0038) | | | | | | | |
| | Group 5 | Group 2 | Group 1 | Group 6 | Group 4 | Control | Group 3 |
| Overall Discoloration - | −1.00 | −0.92 | −0.50 | −0.42 | −0.20 | −0.07 | 0.00 |
| Week 4 (p = 0.0002) | | | | | | | |
| | Group 5 | Group 2 | Group 6 | Group 3 | Group 1 | Group 4 | Control |
| Overall Discoloration - | −1.36 | −1.17 | −1.00 | −0.86 | −0.83 | −0.60 | −0.22 |
| Week 8 (p = <0.0001) | | | | | | | |
| | Group 6 | Group 5 | Group 1 | Group 2 | Group 3 | Group 4 | Control |
| Brightness - Week 4 | −0.83 | −0.79 | −0.75 | −0.75 | −0.71 | −0.40 | −0.34 |
| (p = 0.0192) | | | | | | | |
| | Group 6 | Group 1 | Group 3 | Group 4 | Group 2 | Group 5 | Control |
| Brightness - Week 8 | −1.75 | −1.50 | −1.50 | −1.50 | −1.33 | −1.14 | −0.72 |
| (p = <0.0001) | | | | | | | |
| | Group 3 | Group 5 | Group 2 | Group 6 | Group 1 | Control | Group 4 |
| Clarity - Week 4 | −0.86 | −0.79 | −0.67 | −0.67 | −0.58 | −0.34 | −0.20 |
| (p = 0.0158) | | | | | | | |
| | Group 6 | Group 1 | Group 3 | Group 5 | Group 4 | Group 2 | Control |
| Clarity - Week 8 | −1.42 | −1.17 | −1.14 | −1.14 | −1.10 | −1.00 | −0.43 |
| (p = 0.0007) | | | | | | | |
| | Group 2 | Group 3 | Group 6 | Group 1 | Group 4 | Group 5 | Control |
| Pore Size: Forehead - | −0.75 | −0.64 | −0.58 | −0.50 | −0.40 | −0.36 | −0.22 |
| Week 4 (p = 0.0008) | | | | | | | |
| | Group 2 | Group 3 | Group 1 | Group 6 | Group 4 | Group 5 | Control |
| Pore Size: Forehead - | −1.25 | −1.14 | −1.00 | −1.00 | −0.90 | −0.86 | −0.42 |
| Week 8 (p = <0.0001) | | | | | | | |
| | Group 2 | Group 3 | Group 5 | Group 1 | Group 6 | Control | Group 4 |
| Pore Size: Nose Area - | −0.83 | −0.71 | −0.71 | −0.50 | −0.50 | −0.20 | −0.20 |
| Week 4 (p = <0.0001) | | | | | | | |
| | Group 2 | Group 5 | Group 1 | Group 6 | Group 3 | Control | Group 4 |
| Pore Size: Nose Area - | −1.33 | −1.29 | −1.17 | −1.17 | −1.14 | −0.42 | −0.40 |
| Week 8 (p = <0.0001) | | | | | | | |
| | Group 2 | Group 5 | Group 6 | Group 3 | Group 1 | Control | Group 4 |
| Pore Distribution Week 4 | −0.75 | −0.64 | −0.58 | −0.50 | −0.42 | −0.20 | −0.20 |
| (p = 0.0029) | | | | | | | |
| | Group 2 | Group 5 | Group 6 | Group 1 | Group 3 | Group 4 | Control |
| Pore Distribution - Week 8 | −1.25 | −1.14 | −1.08 | −0.83 | −0.79 | −0.70 | −0.36 |
| (p = 0.0009) | | | | | | | |
| | Group 5 | Group 1 | Group 6 | Group 2 | Group 3 | Group 4 | Control |
| Overall Skin Radiance - | −0.79 | −0.75 | −0.58 | −0.50 | −0.50 | −0.50 | −0.22 |
| Week 4 (p = 0.0016) | | | | | | | |
| | Group 1 | Group 5 | Group 6 | Group 2 | Group 4 | Group 3 | Control |
| Overall Skin Radiance - | −1.50 | −1.50 | −1.50 | −1.33 | −1.30 | −1.00 | −0.49 |
| Week 8 (p = <0.0001) | | | | | | | |
| | Group 3 | Group 6 | Group 5 | Group 4 | Control | Group 1 | Group 2 |
| Erythema - Week 4 | −0.57 | −0.42 | −0.36 | −0.30 | −0.30 | −0.25 | −0.17 |
| (p = 0.0020) | | | | | | | |
| | Group 3 | Group 5 | Group 6 | Control | Group 4 | Group 2 | Group 1 |
| Erythema - Week 8 | −0.64 | −0.43 | −0.42 | −0.31 | −0.30 | −0.17 | 0.00 |
| (p = <0.0001) | | | | | | | |
| | Group 3 | Control | Group 1 | Group 2 | Group 4 | Group 5 | Group 6 |
| Scaling - Week 4 | −0.21 | −0.01 | 0.00 | 0.00 | 0.00 | 0.00 | 0.00 |
| (p = 0.0124) | | | | | | | |
| | Group 3 | Control | Group 1 | Group 2 | Group 4 | Group 5 | Group 6 |
| Scaling - Week 8 | −0.21 | −0.01 | 0.00 | 0.00 | 0.00 | 0.00 | 0.00 |
| (p = 0.0124) | | | | | | | |
| | Group 6 | Group 1 | Control | Group 3 | Group 5 | Group 4 | Group 2 |
| Tightness - Week 4 and | −0.25 | −0.17 | −0.15 | −0.14 | −0.14 | −0.10 | −0.08 |
| Week 8 (p = <0.0001) | | | | | | | |
| | Control | Group 1 | Group 2 | Group 3 | Group 5 | Group 6 | Group 4 |
| Tingling - Week 4 | 0.00 | 0.00 | 0.00 | 0.00 | 0.00 | 0.00 | 0.10 |
| (p = 0.0500) | | | | | | | |
| | Group 4 | Group 3 | Group 6 | Group 1 | Group 5 | Control | Group 2 |
| Corneometer - Week 4 | 33.93 | 24.33 | 18.78 | 14.44 | 14.38 | 8.31 | 7.06 |
| (p = 0.0006) | | | | | | | |
| | Group 4 | Group 3 | Group 6 | Group 5 | Control | Group 2 | Group 1 |
| Corneometer - Week 8 | 20.07 | 13.43 | 10.78 | 10.14 | 6.25 | 3.61 | 0.06 |
| (p = 0.0275) | | | | | | | |
| | Group 6 | Group 5 | Group 4 | Group 2 | Group 3 | Control | Group 1 |
| Chroma Meter: L* - | 1.48 | 0.35 | 0.13 | −0.08 | −0.44 | −0.63 | −3.52 |
| Week 8 (p = 0.0152) | | | | | | | |

At Week 4 and Week 8, subjects completed a Subject Skin Change Evaluation questionnaire and rated their perception of changes in skin condition parameters since the start of the study. Table 7 presents the top box analysis of the Skin Change Evaluation questionnaire for each group. The number of subjects with the specific response is listed, followed by the percentage of the total subject population in parentheses. An asterisk (*) indicates that the proportion of subjects responding positively for a given statement is statistically greater than the proportion of subjects responding negatively. The neutral response option (No Change) was excluded from the analysis for applicable questions.

TABLE 7
RESULTS OF TOP BOX ANALYSIS FOR SUBJECT
SKIN CHANGE EVALUATION QUESTIONNAIRE

| | | Control Product - No Color | |
|---|---|---|---|
| | | Label | |
| | | BETTER: | WORSE: |
| | | Much, Moderately, | Much, Moderately, |
| | | Slightly | Slightly |
| Small, fine lines around the eyes | Week 4 | * 14 (37.8%) | 0 (0.0%) |
| | Week 8 | * 18 (48.6%) | 2 (5.4%) |
| Thick, coarse lines around the eyes | Week 4 | * 12 (32.4%) | 0 (0.0%) |
| | Week 8 | * 16 (44.4%) | 1 (2.8%) |
| How rough skin ‘looks’ | Week 4 | * 19 (51.4%) | 4 (10.8%) |
| | Week 8 | * 20 (54.1%) | 2 (5.4%) |
| How rough skin feels | Week 4 | * 21 (56.8%) | 3 (8.1%) |
| | Week 8 | * 20 (54.1%) | 2 (5.4%) |
| Facial skin discoloration | Week 4 | * 16 (43.2%) | 1 (2.7%) |
| | Week 8 | * 15 (40.5%) | 1 (2.7%) |
| Size of facial pores (forehead/nose) | Week 4 | * 14 (37.8%) | 1 (2.7%) |
| | Week 8 | * 14 (37.8%) | 2 (5.4%) |
| Overall skin radiance | Week 4 | * 20 (54.1%) | 2 (5.4%) |
| | Week 8 | * 22 (59.5%) | 2 (5.4%) |
| | | Group 1 Product - Blue Label | |
| | | BETTER: | WORSE: |
| | | Much, Moderately, | Much, Moderately, |
| | | Slightly | Slightly |
| Small, fine lines around the eyes | Week 4 | 3 (50.0%) | 0 (0.0%) |
| | Week 8 | * 4 (66.7%) | 0 (0.0%) |
| Thick, coarse lines around the eyes | Week 4 | 2 (33.3%) | 0 (0.0%) |
| | Week 8 | 3 (50.0%) | 0 (0.0%) |
| How rough skin ‘looks’ | Week 4 | * 4 (66.7%) | 0 (0.0%) |
| | Week 8 | 3 (50.0%) | 0 (0.0%) |
| How rough skin feels | Week 4 | 2 (33.3%) | 0 (0.0%) |
| | Week 8 | * 4 (66.7%) | 0 (0.0%) |
| Facial skin discoloration | Week 4 | 1 (16.7%) | 0 (0.0%) |
| | Week 8 | 2 (33.3%) | 0 (0.0%) |
| Size of facial pores (forehead/nose) | Week 4 | 1 (16.7%) | 0 (0.0%) |
| | Week 8 | 2 (33.3%) | 0 (0.0%) |
| Overall skin radiance | Week 4 | 2 (33.3%) | 0 (0.0%) |
| | Week 8 | 3 (50.0%) | 0 (0.0%) |
| | | Group 2 Product - Yellow | |
| | | Label | |
| | | BETTER: | WORSE: |
| | | Much, Moderately, | Much, Moderately, |
| | | Slightly | Slightly |
| Small, fine lines around the eyes | Week 4 | 3 (50.0%) | 0 (0.0%) |
| | Week 8 | 3 (50.0%) | 0 (0.0%) |
| Thick, coarse lines around the eyes | Week 4 | 2 (33.3%) | 0 (0.0%) |
| | Week 8 | * 4 (66.7%) | 0 (0.0%) |
| How rough skin ‘looks’ | Week 4 | * 4 (66.7%) | 0 (0.0%) |
| | Week 8 | 3 (50.0%) | 0 (0.0%) |
| How rough skin feels | Week 4 | * 4 (66.7%) | 0 (0.0%) |
| | Week 8 | * 4 (66.7%) | 0 (0.0%) |
| Facial skin discoloration | Week 4 | 3 (50.0%) | 0 (0.0%) |
| | Week 8 | 2 (33.3%) | 0 (0.0%) |
| Size of facial pores (forehead/nose) | Week 4 | 3 (50.0%) | 0 (0.0%) |
| | Week 8 | 2 (33.3%) | 0 (0.0%) |
| Overall skin radiance | Week 4 | * 4 (66.7%) | 0 (0.0%) |
| | Week 8 | * 4 (66.7%) | 0 (0.0%) |
| | | Group 3 Product - Red Label | |
| | | BETTER: | WORSE: |
| | | Much, Moderately, | Much, Moderately, |
| | | Slightly | Slightly |
| Small, fine lines around the eyes | Week 4 | 1 (14.3%) | 0 (0.0%) |
| | Week 8 | 3 (42.9%) | 0 (0.0%) |
| Thick, coarse lines around the eyes | Week 4 | 1 (14.3%) | 0 (0.0%) |
| | Week 8 | 3 (42.9%) | 0 (0.0%) |
| How rough skin ‘looks’ | Week 4 | 2 (28.6%) | 1 (14.3%) |
| | Week 8 | 3 (42.9%) | 0 (0.0%) |
| How rough skin feels | Week 4 | 3 (42.9%) | 1 (14.3%) |
| | Week 8 | 3 (42.9%) | 0 (0.0%) |
| Facial skin discoloration | Week 4 | 2 (28.6%) | 0 (0.0%) |
| | Week 8 | 1 (14.3%) | 0 (0.0%) |
| Size of facial pores (forehead/nose) | Week 4 | 0 (0.0%) | 0 (0.0%) |
| | Week 8 | 2 (28.6%) | 0 (0.0%) |
| Overall skin radiance | Week 4 | 2 (28.6%) | 0 (0.0%) |
| | Week 8 | * 4 (57.1%) | 0 (0.0%) |
| | | Group 4 Product - Green | |
| | | Label | |
| | | BETTER: | WORSE: |
| | | Much, Moderately, | Much, Moderately, |
| | | Slightly | Slightly |
| Small, fine lines around the eyes | Week 4 | 2 (40.0%) | 0 (0.0%) |
| | Week 8 | 1 (20.0%) | 0 (0.0%) |
| Thick, coarse lines around the eyes | Week 4 | 1 (20.0%) | 0 (0.0%) |
| | Week 8 | 1 (20.0%) | 0 (0.0%) |
| How rough skin ‘looks’ | Week 4 | 3 (60.0%) | 0 (0.0%) |
| | Week 8 | * 4 (80.0%) | 0 (0.0%) |
| How rough skin feels | Week 4 | 2 (40.0%) | 1 (20.0%) |
| | Week 8 | 3 (60.0%) | 0 (0.0%) |
| Facial skin discoloration | Week 4 | 2 (40.0%) | 0 (0.0%) |
| | Week 8 | 1 (20.0%) | 0 (0.0%) |
| Size of facial pores (forehead/nose) | Week 4 | 2 (40.0%) | 0 (0.0%) |
| | Week 8 | 2 (40.0%) | 0 (0.0%) |
| Overall skin radiance | Week 4 | 2 (40.0%) | 0 (0.0%) |
| | Week 8 | 3 (60.0%) | 1 (20.0%) |
| | | Group 5 Product - | |
| | | Yellow with Black Line Label | |
| | | BETTER: | WORSE: |
| | | Much, Moderately, | Much, Moderately, |
| | | Slightly | Slightly |
| Small, fine lines around the eyes | Week 4 | * 4 (57.1%) | 0 (0.0%) |
| | Week 8 | * 5 (71.4%) | 0 (0.0%) |
| Thick, coarse lines around the eyes | Week 4 | 3 (42.9%) | 1 (14.3%) |
| | Week 8 | 3 (42.9%) | 0 (0.0%) |
| How rough skin ‘looks’ | Week 4 | * 4 (57.1%) | 0 (0.0%) |
| | Week 8 | * 5 (71.4%) | 0 (0.0%) |
| How rough skin feels | Week 4 | 3 (42.9%) | 0 (0.0%) |
| | Week 8 | * 4 (57.1%) | 0 (0.0%) |
| Facial skin discoloration | Week 4 | 3 (42.9%) | 0 (0.0%) |
| | Week 8 | * 5 (71.4%) | 0 (0.0%) |
| Size of facial pores (forehead/nose) | Week 4 | 3 (42.9%) | 1 (14.3%) |
| | Week 8 | 3 (42.9%) | 0 (0.0%) |
| Overall skin radiance | Week 4 | 4 (57.1%) | 1 (14.3%) |
| | Week 8 | * 7 (100.0%) | 0 (0.0%) |
| | | Group 6 Product - | |
| | | Red with Black Line Label | |
| | | BETTER: | WORSE: |
| | | Much, Moderately, | Much, Moderately, |
| | | Slightly | Slightly |
| Small, fine lines around the eyes | Week 4 | 1 (16.7%) | 1 (16.7%) |
| | Week 8 | 2 (33.3%) | 0 (0.0%) |
| Thick, coarse lines around the eyes | Week 4 | 1 (16.7%) | 0 (0.0%) |
| | Week 8 | 2 (33.3%) | 0 (0.0%) |
| How rough skin ‘looks’ | Week 4 | 3 (50.0%) | 0 (0.0%) |
| | Week 8 | 3 (50.0%) | 0 (0.0%) |
| How rough skin feels | Week 4 | * 4 (66.7%) | 0 (0.0%) |
| | Week 8 | 3 (50.0%) | 0 (0.0%) |
| Facial skin discoloration | Week 4 | 3 (50.0%) | 0 (0.0%) |
| | Week 8 | 2 (33.3%) | 0 (0.0%) |
| Size of facial pores (forehead/nose) | Week 4 | 3 (50.0%) | 0 (0.0%) |
| | Week 8 | 2 (33.3%) | 0 (0.0%) |
| Overall skin radiance | Week 4 | * 4 (66.7%) | 0 (0.0%) |
| | Week 8 | 3 (50.0%) | 0 (0.0%) |

### Example 10

### Summary of Clinical Grading and Instrumentation Results

At each visit, subjects participated in the following clinical grading and instrumentation procedures on the right and left sides of the face:
Clinical grading of the following efficacy/performance parameters: fine wrinkles (periocular), coarse wrinkles (periocular), skin texture (cheeks), overall discoloration, brightness (cheeks), clarity of skin, pore size (forehead and nose area), pore distribution/structure, and overall skin radiance.
Clinical grading of the following irritation/safety parameters: erythema, edema, scaling, burning, stinging, itching, tightness, tingling)
Triplicate Skin surface hydration measurements (Corneometer® CM 825, Courage+Khazaka, Germany) measurements were taken on the cheeks to assess moisturization
Triplicate skin luminance measurements (Chroma Meter CR400, Konica-Minolta) were taken on pigmented lesions to instrumentally assess changes in skin color/tone
Skin visco-elasticity measurements (Cutometer® SEM 575, Courage+Khazaka, Germany) were taken on the cheeks to assess the visco-elastic properties of the skin

The following table illustrates the statistically significant differences compared to Baseline for the clinical grading and instrumentation parameters. Significant differences compared to Baseline are indicated using an up or down arrow. Parameters with no significant differences are not listed.

TABLE 8

| | | | | | | | | | | | Group 5 - | | Group 6 - | |
|---|---|---|---|---|---|---|---|---|---|---|---|---|---|---|
| | Control | | Group 1 - | | Group 2 - | | Group 3 - | | Group 4 - | | Yellow | | Red | |
| | Product | | Blue | | Yellow | | Red | | Green | | & Black | | & Black | |
| | W4 | W8 | W4 | W8 | W4 | W8 | W4 | W8 | W4 | W8 | W4 | W8 | W4 | W8 |
| EFFICACY GRADING | | | | | | | | | | | | | | |
| Fine Wrinkles | | [symbol] | [symbol] | [symbol] | [symbol] | [symbol] | | [symbol] | | [symbol] | | [symbol] | [symbol] | [symbol] |
| Skin Texture | [symbol] | [symbol] | | [symbol] | | [symbol] | | [symbol] | | | [symbol] | [symbol] | [symbol] | [symbol] |
| Tactile Roughness | [symbol] | [symbol] | [symbol] | [symbol] | [symbol] | [symbol] | [symbol] | [symbol] | [symbol] | [symbol] | [symbol] | [symbol] | [symbol] | [symbol] |
| Overall Discoloration | | [symbol] | [symbol] | [symbol] | [symbol] | [symbol] | | [symbol] | | | [symbol] | [symbol] | | [symbol] |
| Brightness | [symbol] | [symbol] | [symbol] | [symbol] | [symbol] | [symbol] | [symbol] | [symbol] | | [symbol] | [symbol] | [symbol] | [symbol] | [symbol] |
| Clarity of Skin | [symbol] | [symbol] | [symbol] | [symbol] | [symbol] | [symbol] | [symbol] | [symbol] | | | [symbol] | [symbol] | | |
| Pore Size - forehead | [symbol] | [symbol] | | [symbol] | [symbol] | [symbol] | [symbol] | [symbol] | | | | [symbol] | [symbol] | [symbol] |
| Pore Size - nose area | [symbol] | [symbol] | | [symbol] | [symbol] | [symbol] | [symbol] | [symbol] | | | [symbol] | [symbol] | | [symbol] |
| Pore Distribution | [symbol] | [symbol] | | | [symbol] | [symbol] | [symbol] | [symbol] | | | [symbol] | [symbol] | [symbol] | [symbol] |
| Overall Skin Radiance | [symbol] | [symbol] | [symbol] | [symbol] | [symbol] | [symbol] | [symbol] | [symbol] | | [symbol] | [symbol] | [symbol] | [symbol] | [symbol] |
| IRRITATION GRADING | | | | | | | | | | | | | | |
| Erythema | [symbol] | [symbol] | | | | | [symbol] | [symbol] | | | [symbol] | [symbol] | [symbol] | [symbol] |
| Tightness | [symbol] | [symbol] | | | | | | | | | | | | |
| CORNEOMETER | [symbol] | [symbol] | | | | | [symbol] | | [symbol] | [symbol] | | | [symbol] | |
| CHROMA METER | | | | | | | | | | | | | | |
| L* | | | | [symbol] | | | | | | | | | | |
| a* | [symbol] | [symbol] | | | [symbol] | | | [symbol] | | | | | [symbol] | [symbol] |
| b* | | | | [symbol] | | | | | | | | | | |
| CUTOMETER | | | | | | | | | | | | | | |
| Biological Elasticity | | | | | | | [symbol] | [symbol] | | | [symbol] | | | |
| Extensibility | | | | | | | | | | | | | | [symbol] |
| Pure Elasticity | | | [symbol] | | | | [symbol] | | [symbol] | | [symbol] | | | |
| Resiliency | | | | | | | [symbol] | [symbol] | | | [symbol] | [symbol] | | |

Comparisons, based on the average change from Baseline, were made among the seven treatments using analysis of variance (ANOVA) with paired comparisons (Fisher's LSD). Results of the ANOVA comparisons showed significant differences among the treatments for all efficacy grading parameters (with the exception of periocular coarse wrinkles), some irritation parameters (erythema, scaling, tightness, tingling), and for some instrumentation parameters (Corneometer, Chroma Meter L*).

### OVERALL CONCLUSIONS

The data provided herein provide important results that illustrate that, although the criteria evaluated for efficacy and performance showed positive benefits with the control composition alone, only the test formulae were effective in increasing the viscoelasticity resiliency and hydration of the test subjects skin. These important findings demonstrate that, while compounds contained in the control formula and/or that may be normal constituents of cosmetics or skin care products have a positive effect on visible skin attributes, including for example, roughness and clarity, it is the arNOX inhibitory compounds that are capable of increasing elasticity and resiliency in the skin after only four weeks and throughout the eight week trial period. Further, it is important to note that while the control showed a significant increase in hydration or Corneometer® measurements, the absolute increase in hydration was only 16.3 and 12.2 percent at the 4 and 8 week time points respectively. In contrast, test groups 1, 3, 4, 5, and 6 showed increases in hydration that were 27.9, 57.8, 92.2, 27.7, and 38.8 percent improved respectively at the 4 week time point and 31.9, 54.5, 19.5 and 22.3 for groups 3, 4, 5, and 6 respectively at the 8 week time point. Further, while the control group had no real effect on elasticity, all of the test formulations showed a tendency to increase skin elasticity, some with exceptional results. For example, Group 3 (Shizandra chinensis) showed a remarkable increase in all measures of elasticity ranging from 14.4 to a low of 5.9% in just 4 weeks. Together with the almost 60% increase in hydration at 4 weeks and approximately 30% hydration at 8 weeks these are positive effects that could not have been predicted or anticipated. Further, it is important to point out that, as shown in Table 1, Schisandra chinensis showed a 100% inhibition of arNOX. These data illustrate an important correlation with the results disclosed herein in inhibiting or ameliorating the effects of aging on the skin and further and illustrates the value of such agents in preparations, particularly cosmetic preparations as part of a daily use regimen.

While this invention has been described in conjunction with the various exemplary embodiments outlined above, various alternatives, modifications, variations improvements, and/or substantial equivalents, whether known or that are or may be presently unforeseen, may become apparent to those having at least ordinary skill in the art. Accordingly, the exemplary embodiments according to this invention, as set forth above, are intended to be illustrative not limiting. Various changes may be made without departing from the spirit and scope of the invention. Therefore, the invention is intended to embrace all known or later-developed alternatives, modifications, variations, improvements, an/or substantial equivalents of these exemplary embodiments.

1. A topical composition useful for ameliorating the effects of aging comprising:
an effective amount of at least one arNOX inhibitory agent, wherein the arNOX inhibitory agent is effective in decreasing the effects of aging.

2. The topical composition of claim 1, wherein the composition further includes a cosmetically or pharmaceutically acceptable carrier.

3. The topical composition of claim 1, wherein the arNOX inhibitory agent has a greater than 10% inhibition of arNOX.

4. The topical composition of claim 1, wherein the arNOX inhibitory agent is present in a plant extract.

5. The topical composition of claim 4, wherein the plant is selected from broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta or olive.

6. The topical composition of claim 5, wherein the Lonicera is Lonicera japonica or Lonicera caprifolium.

7. The topical composition of claim 1, wherein the arNOX inhibitory agent is β-carotene or astaxanthin.

8. The topical composition of claim 1, wherein the composition is administered as a cream, a milk, a lotion, a gel, a suspension of lipid or polymeric microspheres or nanospheres or vesicles, a soap, a shampoo or a sunscreen.

9. The topical composition of claim 1, wherein the effects of aging comprise: lines, wrinkles, hyperpigmentation, dehydration, loss of elasticity, angioma, dryness, itching, telangietasias, actinic purpura, seborrheic keratoses, lack of hydration, decrease in collagen or actinic keratoses.

10. The topical composition of claim 1, wherein the arNOX inhibitory agent is provided at a concentration of between about 5 μg/ml to about 500 μg/ml.

11. The topical composition of claim 10, wherein the arNOX inhibitory agent is provided at a concentration of between about 15 μg/ml to about 100 μg/ml.

12. A cosmetic composition for ameliorating the effects of aging comprising:
a cosmetically effective amount of at least one arNOX inhibitory agent wherein the arNOX inhibitory agent is effective in decreasing the effects of aging upon the skin.

13. The composition of claim 12, wherein the arNOX inhibitory agent is provided in a cosmetic preparation at a concentration of between about 5 μg/ml to about 500 μg/ml.

14. The composition of claim 13, wherein the arNOX inhibitory agent is provided in a cosmetic preparation at a concentration of between about 15 μg/ml to about 100 μg/ml.

15. The cosmetic composition of claim 12, wherein the arNOX inhibitory agent is present in a plant extract.

16. The cosmetic composition of claim 15, wherein the plant comprises broccoli, shitake, coleus rosemary, lotus, artichoke, sea rose tangerine, Oenothera biennis, astaxanthin, red orange, Schisandra chinensis, Lonicera, Fagopyrum, carrot, Narcissus tazetta or olive.

17. The cosmetic composition of claim 16, wherein the Lonicera is Lonicera japonica or Lonicera caprifolium.

18. The cosmetic composition of claim 12, wherein the arNOX inhibitory agent is β-carotene or astaxanthin.

19. The cosmetic composition of claim 12, wherein the composition is applied as a cream, a milk, a lotion, a gel, a suspension of lipid or polymeric microspheres or nanospheres or vesicles, a soap, a shampoo or a sunscreen.

20. The cosmetic composition of claim 12, wherein the effects of aging comprise: lines, wrinkles, hyperpigmentation, dehydration, loss of elasticity, angioma, dryness, itching, telangietasias, actinic purpura, seborrheic keratoses, lack of hydration, decrease in collagen or actinic keratoses.

21. The cosmetic composition of claim 12, formulated as a cream, a milk, a lotion, a gel, a suspension of lipid or polymeric microspheres or nanospheres or vesicles, a soap or a shampoo.

22. A cosmetic method for ameliorating the effects of aging comprising applying to the skin a cosmetic composition comprising:
an effective amount of an arNOX inhibitor,
wherein at least one arNOX mediated effect of aging is inhibited.

23. The method of claim 22, wherein the arNOX inhibitor is derived from a plant extract.

24. The method of claim 23, wherein the plant extract comprises carrot, olive, broccoli, shitake, coleus, rosemary, lotus, artichoke, sea rose tangerine, Oenothera biennis, red orange, Schisandra chinensis, Lonicera, Fagopyrum or Narcissus tazetta.

25. The cosmetic method of claim 22, wherein the arNOX inhibitor is purified from a plant extract.

26. The cosmetic method of claim 25, wherein the purified arNOX inhibitor is β-carotene or astaxanthin.

27. The cosmetic method of claim 22, wherein the arNOX inhibitor is provided together with a cosmetically acceptable carrier.

28. The cosmetic method of claim 22, wherein the effects of aging comprise: lines, wrinkles, hyperpigmentation, dehydration, loss of elasticity, angioma, dryness, itching, telangietasias, actinic purpura, seborrheic keratoses, lack of hydration, decrease in collagen or actinic keratoses.

29. The cosmetic method of claim 22, wherein the arNOX inhibitor is applied at least once a day.

30. The cosmetic method of claim 22, wherein the arNOX inhibitory agent is provided in a cosmetic preparation at a concentration of between about 5 μg/ml to about 500 μg/ml.

31. The cosmetic method of claim 30, wherein the arNOX inhibitory agent is provided in a cosmetic preparation at a concentration of between about 15 to about 100 μg/ml.

32. The cosmetic method of claim 22, wherein the composition is administered as a cream, a milk, a lotion, a gel, a suspension of lipid or polymeric microspheres or nanospheres or vesicles, a soap, a shampoo or a sunscreen.

33. A kit for applying a cosmetic useful in ameliorating the effects of aging comprising:
at least one arNOX inhibitory plant extract; and
instruction for use.

31. The kit of claim 33, further comprising a cosmetic preparation suitable as a carrier for the at least one arNOX inhibitory plant extract.

35. The cosmetic composition of claim 12, wherein the composition further includes a cosmetically acceptable carrier.

36. The cosmetic composition of claim 12, wherein the arNOX inhibitory agent has a greater than 10% inhibition of arNOX.

37. The topical composition of claim 4, wherein the plant is Narcissus tazetta.

38. The topical composition of claim 37, further comprising Schisandra chinensis.
